(19) United States
(12) Patent Application Publication
(10) Pub. No.: US 2022/0275053 A1
(43) Pub. Date: Sep. 1, 2022

(54) MODIFIED MULTIMERIC BICYCLIC PEPTIDE LIGANDS

(71) Applicant: BicycleTx Limited, Cambridge (GB)

(72) Inventors: Punit UPADHYAYA, Lexington, MA (US); Gemma Elizabeth MUDD, Cambridge (GB); Kevin MCDONNELL, Lexington, MA (US)

(21) Appl. No.: 17/630,754

(22) PCT Filed: Aug. 13, 2020
(86) PCT No.: PCT/GB2020/051923

§ 371 Date: Jan. 27, 2022

Related U.S. Application Data
(60) Provisional application No. 62/885,947, filed on Aug. 13, 2019.

(30) Foreign Application Priority Data
Jul. 30, 2020 (GB) PCT/GB2020/051831

(51) Int. Cl. C07K 14/705; A61K 47/64; C07K 7/06; A61P 35/00

(52) U.S. Cl. CPC C07K 14/70578; A61K 47/64; C07K 14/70596; C07K 7/06; A61P 35/00; A61K 38/00

(57) ABSTRACT

The present invention relates to multimers of polypeptides which are covalently bound to molecular scaffolds such that two or more peptide loops are subtended between attachment points to the scaffold, characterised in that said multimeric binding complex additionally comprises a modifier group conjugated thereto. The invention also describes the multimerization of polypeptides through various chemical linkers and hinges of various lengths and rigidity using different sites of attachments within polypeptides. In particular, the invention describes multimers of peptides which are high affinity binders and activators of CD137. The invention also includes drug conjugates comprising said multimeric binding complexes, conjugated to one or more effector and/or functional groups, to pharmaceutical compositions comprising said multimeric binding complexes and drug conjugates and to the use of said multimeric binding complexes and drug conjugates in preventing, suppressing or treating a disease or disorder mediated by CD137 and to the use in an analytical method (i.e. as a tracer or a tag).

### FIELD OF THE INVENTION

The present invention relates to multimers of polypeptides which are covalently bound to molecular scaffolds such that two or more peptide loops are subtended between attachment points to the scaffold, characterised in that said multimeric binding complex additionally comprises a modifier group conjugated thereto. The invention also describes the multimerization of polypeptides through various chemical linkers and hinges of various lengths and rigidity using different sites of attachments within polypeptides. In particular, the invention describes multimers of peptides which are high affinity binders and activators of CD137. The invention also includes drug conjugates comprising said multimeric binding complexes, conjugated to one or more effector and/or functional groups, to pharmaceutical compositions comprising said multimeric binding complexes and drug conjugates and to the use of said multimeric binding complexes and drug conjugates in preventing, suppressing or treating a disease or disorder mediated by CD137 and to the use in an analytical method (i.e. as a tracer or a tag).

### BACKGROUND OF THE INVENTION

Protein-protein interactions are important regulators of cellular functions. These interactions typically involve large surface areas and as such can neither be easily inhibited nor mimicked using typical small molecule therapeutic agents. Additionally, many important receptor classes (receptor tyrosine kinases, cytokine receptors, tumor necrosis factor (TNF) receptors, T-cell receptors and G-protein coupled receptors) require oligomerization of receptor monomer units in a particular orientation to activate the receptor signaling pathway. Recombinant proteins such as monoclonal antibodies and fusion proteins (e.g. ligand-Fc fusions) are able to bind and induce oligomerization of such receptors due to high affinity and large interaction surface areas with the potential for multivalent binding. However, large proteins are inefficient at penetrating into tissues and may not be an ideal therapeutic modality for modulating receptors, especially those found on cells that are poorly vascularized or surrounded by barriers to penetration, such as the stromal barrier found in pancreatic cancer. Small synthetic and modular therapeutic modalities with a larger interaction surface than small molecules will be ideal for bypassing the penetration barrier and activating target receptors by oligomerization.

The recent success of immune checkpoint inhibitors, such as anti-PD-1 and anti-PD-L1 antibodies in treating various types of cancers have boosted the interest in molecules that activate co-stimulatory targets, including CD137 on T cells. CD137 (4-1BB/TNFRSF9) belongs to the TNF receptor superfamily and provides costimulatory signaling for T cells.

Inducible CD137 expression is found on activated T-, B-, dendritic and natural killer (NK) cells. Stimulation of CD137 by its natural ligand, CD137L, or by agonistic antibody induces vigorous T-cell proliferation and prevents activation-induced cell death. 4-1BB forms a heterotrimer complex consisting of two TNF-receptor associated factor TRAF-2 complexes in conjunction with TRAF-1. This interaction, through leukocyte specific protein-1 (LSP-1), potentiates signaling through JNK and ERK pathways as well as through β-catenin and AKT. These signaling pathways converge on the master transcription factor NF-κB to regulate 4-1BB signaling, as well as effector immune responses.

Agonistic anti-CD137 antibodies have shown potent, often curative anti-tumor activity in mouse models. Its anti-tumor activity is even further boosted in combination with an anti-PD-1 or anti-CTLA-4 antibody. These effects are mainly mediated by cytotoxic T cells and generate long lasting, memory responses. Two human anti-CD137 antibodies are currently undergoing clinical testing: urelumab has shown single agent, partial responses in melanoma, however hepatoxicity was observed at doses≥1 mg/kg and as a result, it is being combined with other immunotherapies at a suboptimal dose of 0.1 mg/kg; utolimumab is also being evaluated in solid tumors in combination with other immunotherapies, but while hepatotoxicity was not observed up to 5 mg/kg, it has little or no single agent activity.

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred-square angstrom, as for example the cyclic peptide CXCR4 antagonist CVX15 (400 Å2; Wu et al. (2007), Science 330, 1066-71), a cyclic peptide with the Arg-Gly-Asp motif binding to integrin αVβ3 (355 Å2) (Xiong et al. (2002), Science 296 (5565), 151-5) or the cyclic peptide inhibitor upain-1 binding to urokinase-type plasminogen activator (603 Å2; Zhao et al. (2007), J Struct Biol 160 (1), 1-10).

Bicycles® are a novel therapeutic class of fully synthetic, constrained bicyclic peptides that have high affinity and exquisite target specificity unachievable with conventional small molecule approaches. The Bicycle® platform uses phage display to rapidly identify and optimize binders that can then be readily chemically optimized to tune affinity and physicochemical properties. Their small size (1.5-2 kDa) delivers advantages in tumor penetration and rapid renal elimination avoids liver and gastrointestinal toxicity often associated with other drug modalities, including certain antibodies. Bicycle® CD137 agonists with rapid renal clearance and lacking Fc receptor interaction could induce anti-tumor activity while avoiding liver toxicity.

There is a need to provide alternative bicyclic peptides with modified properties to be able to detect, trace, tag or alter the pharmacokinetic profile of said bicyclic peptide.

### SUMMARY OF THE INVENTION

According to a first aspect of the invention, there is provided a multimeric binding complex which comprises at least two bicyclic peptide ligands, wherein said peptide ligands may be the same or different, each of which comprises a polypeptide comprising at least three reactive groups, separated by at least two loop sequences, and a molecular scaffold which forms covalent bonds with the reactive groups of the polypeptide such that at least two polypeptide loops are formed on the molecular scaffold, characterised in that said multimeric binding complex additionally comprises a modifier group conjugated thereto.

According to a further aspect of the invention, there is provided a drug conjugate comprising a multimeric binding complex as defined herein conjugated to one or more effector and/or functional groups.

According to a further aspect of the invention, there is provided a pharmaceutical composition comprising a multimeric binding complex or a drug conjugate as defined herein in combination with one or more pharmaceutically acceptable excipients.

According to a further aspect of the invention, there is provided a multimeric binding complex or drug conjugate as defined herein for use in preventing, suppressing or treating a disease or disorder, such as a disease or disorder mediated by CD137.

According to a further aspect of the invention, there is provided the use of a multimeric binding complex as defined herein in an analytical method (i.e. as a tracer or a tag).

### BRIEF DESCRIPTION OF THE FIGURES

FIG. 1: CD137 Reporter cell activity assay data for BCY7340, BCY9931 and BCY9932 compared to CD137L.

FIG. 2: CD137 Reporter cell Washout assay for BCY9931 and BCY9932 compared to CD137L.

FIG. 3: (A) Binding of BCY9931, BCY9932 and BCY7340 to purified —CD8+/CD137+ T cells isolated from human PBMCs. BCY0215 is a fluorescent EphA2 bicyclic peptide monomer that was used as a negative control for binding. (B) Binding of BCY12239 and BCY11856 to CD3+/CD137+ primary human immune cells. BCY11856 was designed as a non-binding fluorescent multimer.

FIG. 4: Binding of BCY9931, BCY9932 and BCY7340 to purified CD8+/CD137+ T cells isolated from cynomolgus monkey PBMCsBCY0215 is a fluorescent EphA2 bicyclic peptide monomer that was used as a negative control for binding.

FIG. 5: Receptor occupancy assay to measure levels of free CD137 receptor on T-cells upon treatment with BCY8945, BCY7842, BCY7839 and BCY11451 (non-modified multimers) with BCY7340 as the labelling reagent. BCY11451 was synthesized as a non-binding multimer.

FIG. 6: Cell binding of fluorescently labelled CD137 dimer (BCY15416) to CD137+ or CD137− cells in either A) CD4+ T-cells or B) CD8+ T-cells subpopulations.

FIG. 7: Receptor occupancy assay to measure levels of free CD137 receptor on T-cells upon treatment with BCY12491 (EphA2/CD137 heterotandem) and BCY12797 (non-binding control) with BCY15416 as the labelling reagent.

### DETAILED DESCRIPTION OF THE INVENTION

According to a first aspect of the invention, there is provided a multimeric binding complex which comprises at least two bicyclic peptide ligands, wherein said peptide ligands may be the same or different, each of which comprises a polypeptide comprising at least three reactive groups, separated by at least two loop sequences, and a molecular scaffold which forms covalent bonds with the reactive groups of the polypeptide such that at least two polypeptide loops are formed on the molecular scaffold, characterised in that said multimeric binding complex additionally comprises a modifier group conjugated thereto.

The present invention describes modified multimeric binding complexes wherein the presence of said modifier group provides a variety of advantages over the non-modified multimers thereof. For example, such modified multimeric complexes are able to function as tracers, tags or even alter their pharmacokinetic profile, for analytical methods or enhanced therapeutic methods. Thus, according to the invention, there is provided the use of a multimeric binding complex as defined herein in an analytical method (i.e. as a tracer or a tag). According to the invention, there is also provided the use of a multimeric binding complex as defined herein in a therapeutic method (i.e. as a therapeutic with an enhanced pharmacokinetic profile).

References herein to the term “modifier” refer to any group which is capable of modifying the nature and/or characteristics of the non-modified multimeric binding complex.

In one embodiment, the modifier group comprises a tracer molecule, a detectable moiety or a lipid.

In one embodiment, the modifier group comprises a tracer molecule. Such tracer molecules find particular utility in the ability to trace, tag or flag the presence of a multimeric binding complex in either an in vitro or in vivo experiment. Alternatively, the presence of such a tracer molecule is able to identify or determine binding partners with said multimeric binding complexes. In a further embodiment, the tracer molecule is a fluorophore. In a yet further embodiment, the tracer molecule is a fluorophore selected from fluorescein, Alexa Fluor™ 488, cyanine-5 and BODIPY™ FL.

In an alternative embodiment, the modifier group comprises a detectable moiety. In a further embodiment, the modifier group comprises a binding detectable moiety. In a yet further embodiment, the modifier group comprises a biotin containing moiety such as a biotin containing and pegylated moiety, such as Biotin-Peg4 and Biotin-Peg12.

In an alternative embodiment, the modifier group comprises a lipid. The presence of such lipid molecules provide the advantage of modifying the pharmacokinetic profile of a multimeric binding complex for example by modifying the half-life of said multimeric binding complex. In a further embodiment, the lipid is a palmitoyl containing moiety.

In one embodiment, the modifier group is selected from a fluorophore, biotin or a lipid.

Without being bound by theory it is believed that multimerized bicyclic peptides are able to activate receptors by homo-crosslinking more than one of the same receptor. Thus, in one embodiment, said bicyclic peptide ligands are specific for the same target. In a further embodiment, the multimeric binding complex comprises at least two identical bicyclic peptide ligands. By “identical” it is meant bicyclic peptides having the same amino acid sequence, most critically the same amino acid sequence refers to the binding portion of said bicyclic peptide (for example, the sequence may vary in attachment position). In this embodiment, each of the bicyclic peptides within the multimeric binding complex will bind exactly the same epitope upon the same target—the resultant target bound complex will therefore create a homodimer (if the multimeric complex comprises two identical bicyclic peptides), homotrimer (if the multimeric complex comprises three identical bicyclic peptides) or homotetramer (if the multimeric complex comprises four identical bicyclic peptides), etc.

In an alternative embodiment, the multimeric binding complex comprises at least two differing bicyclic peptide ligands. By “differing” it is meant bicyclic peptides having a different amino acid sequence. In this embodiment, the differing bicyclic peptide ligands within the multimeric binding complex will bind to different epitopes on the same target—the resultant target bound complex will therefore create a biparatopic (if the multimeric complex comprises two differing bicyclic peptides), triparatopic (if the multimeric complex comprises three differing bicyclic peptides) or tetraparatopic (if the multimeric complex comprises four differing bicyclic peptides), etc.

Without being bound by theory it is believed that multimerized bicyclic peptides are able to activate receptors by hetero-crosslinking differing targets, such as differing target receptors. Thus, in one embodiment, said bicyclic peptide ligands are specific for different targets. It will be appreciated that in this embodiment, the multimeric binding complex comprises at least two differing bicyclic peptide ligands (i.e. bicyclic peptide ligands having differing amino acid sequences). In this embodiment, each of the bicyclic peptides within the multimeric binding complex will bind a differing epitope upon a different target—the resultant target bound complex will therefore create a bispecific multimeric binding complex (if the multimeric complex comprises two differing bicyclic peptides), trispecific multimeric binding complex (if the multimeric complex comprises three differing bicyclic peptides), tetraspecific multimeric binding complex (if the multimeric complex comprises four differing bicyclic peptides), etc.

It will be appreciated that the multimeric binding complexes of the invention may be designed to be capable of binding to a range of different targets, such as receptors. Suitable examples include any target (i.e. receptor) involved in a cancer, such as members of the TNF receptor superfamily (i.e. CD137), receptor tyrosine kinase (RTK), Ig domain receptors (immune checkpoint) etc. It will be appreciated that for the bi-, tri- and tetra-specific multimeric binding complexes referred to hereinbefore the bicyclic peptides may bind to targets on at least two differing cells (such as T, NK or other immune cells).

The bicyclic peptides within the multimeric binding complexes of the invention may be assembled via a number of differing options. For example, there may be a central hinge or branching moiety with spacer or arm elements radiating from said hinge or branch point each of which will contain a bicyclic peptide. Alternatively, it could be envisaged that a circular support member may hold a number of inwardly or outwardly projecting bicyclic peptides.

In one embodiment, each bicyclic peptide ligand is connected to a central hinge moiety (CHM) by a spacer group (S₁ or S₂) and the modifier group is conjugated directly to one of the bicyclic peptides within the multimeric binding complex. Thus, in one embodiment, the multimeric binding complex comprises a compound of formula (I):

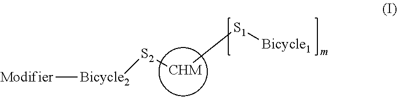

wherein CHM represents a central hinge moiety;
S₁ and S₂ represent spacer groups;
Bicycle₁ and Bicycle₂ represent bicyclic peptide ligands as defined herein;
m represents an integer selected from 1 to 9; and
Modifier represents the modifier group as defined herein.

In an alternative embodiment, each bicyclic peptide ligand is connected to a central hinge moiety (CHM) by a spacer group (S₁) and the modifier group is also connected to the central hinge moiety (CHM) by a further spacer group (S₂). Thus, in one embodiment, the multimeric binding complex comprises a compound of formula (II):

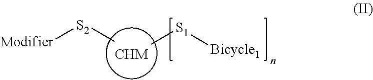

wherein CHM represents a central hinge moiety;
S₁ and S₂ represent spacer groups;
Bicycle₁ represents a bicyclic peptide ligand as defined herein;
n represents an integer selected from 2 to 10; and
Modifier represents the modifier group as defined herein.

In one embodiment, m represents an integer selected from 2 to 9. In a further embodiment, m represents an integer selected from 2 or 3.

When m represents 3, it will be appreciated that the central hinge moiety will require 4 points of attachment (i.e. 3 points of attachment to the 3 Bicycle₁ moieties and 1 point of attachment with the single Bicycle₂ moiety).

In a yet further embodiment, m represents 3. When m represents 3, it will be appreciated that the central hinge moiety will require 4 points of attachment (i.e. 3 points of attachment to the 3 Bicycle₁ moieties and 1 point of attachment with the single Bicycle₂ moiety).

Thus, in one embodiment, m represents 3 and CHM is a motif of formula (A):

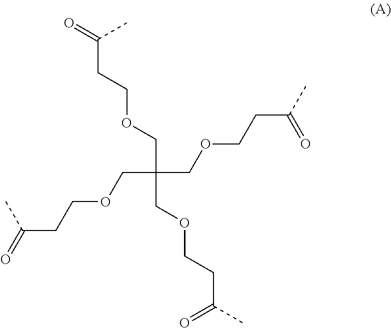

wherein “-----” represents the point of attachment to each spacer group (S₁ or S₂).

In one embodiment, n represents an integer selected from 2 to 9. In a further embodiment, n represents an integer selected from 2 or 3.

When n represents 3, it will be appreciated that the central hinge moiety will require 4 points of attachment (i.e. 3 points of attachment to the 3 Bicycle₁ moieties and 1 point of attachment with the modifier group). Thus, in one embodiment, n represents 3 and CHM is a motif of formula (A):

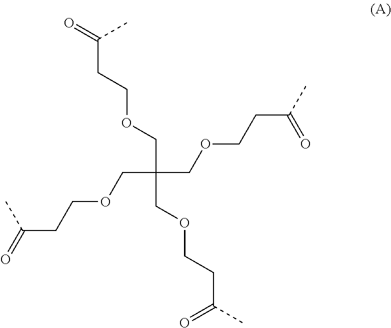

wherein “-----” represents the point of attachment to each spacer group (S₁ or S₂) of the modifier group or Bicycle.

In an alternative embodiment, n represents 3 and CHM is a motif of formula (B):

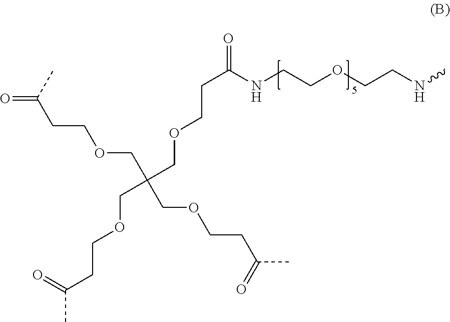

wherein “-----” represents the point of attachment to the spacer group; and
“ [symbol] ” represents the point of attachment to the modifier group.

When n represents 2, it will be appreciated that the central hinge moiety will require 3 points of attachment (i.e. 2 points of attachment to the 2 Bicycle₁ moieties and 1 point of attachment with the modifier group). Thus, in one embodiment, n represents 2 and CHM is a motif of formula (C):

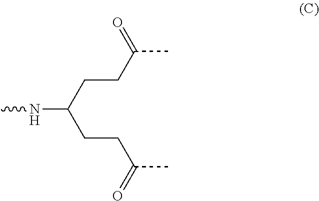

wherein “-----” represents the point of attachment to the spacer group; and
“ [symbol] ” represents the point of attachment to the modifier group.

In an alternative embodiment, n represents 2, and CHM is a motif of formula (D):

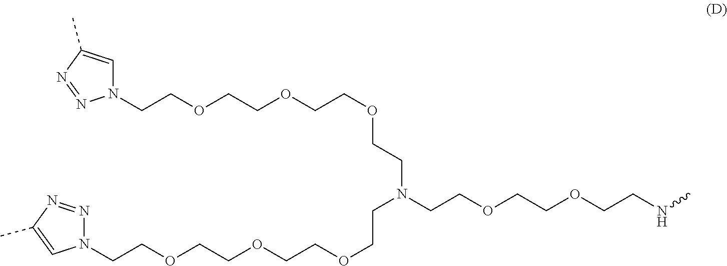

wherein “-----” represents the point of attachment to the spacer group; and
“ [symbol] ” represents the point of attachment to the modifier group.

In one particular embodiment, CHM is:

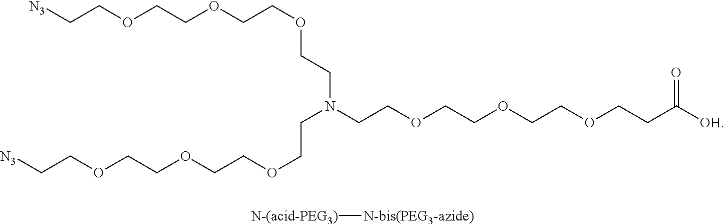

It will be readily apparent to the skilled person how alternative central hinge moieties may be constructed depending upon the values of m and n.

It will be appreciated that the spacers (S₁ and S₂) may be any suitable construction to link the bicyclic peptide central hinge moiety to the bicyclic peptide. In one embodiment, the spacers (S₁ and S₂) comprise a triazolyl moiety. The advantage of this embodiment is that the triazolyl moiety may be incorporated within the synthesis using commonly available “click” chemistry. Examples of suitable spacer (S₁ and S₂) groups include one or more PEG moieties, peptide sequences, carbohydrates, lipids and the like.

In a further embodiment, the spacers (S₁ and S₂) comprise one or more PEG moieties. References herein to “PEG” refer to a linear polymer with a regular repeat unit of the general structure: (CH₂CH₂O)ₙ— (where n represents any number, such as 1 to 30).

Thus, in a further embodiment, the spacers (S₁ and S₂) are selected from any one of spacers S_{A}, S_{B}, S_{C}, S_{D}, S_{E}, S_{F}, S_{G} and S_{H}:

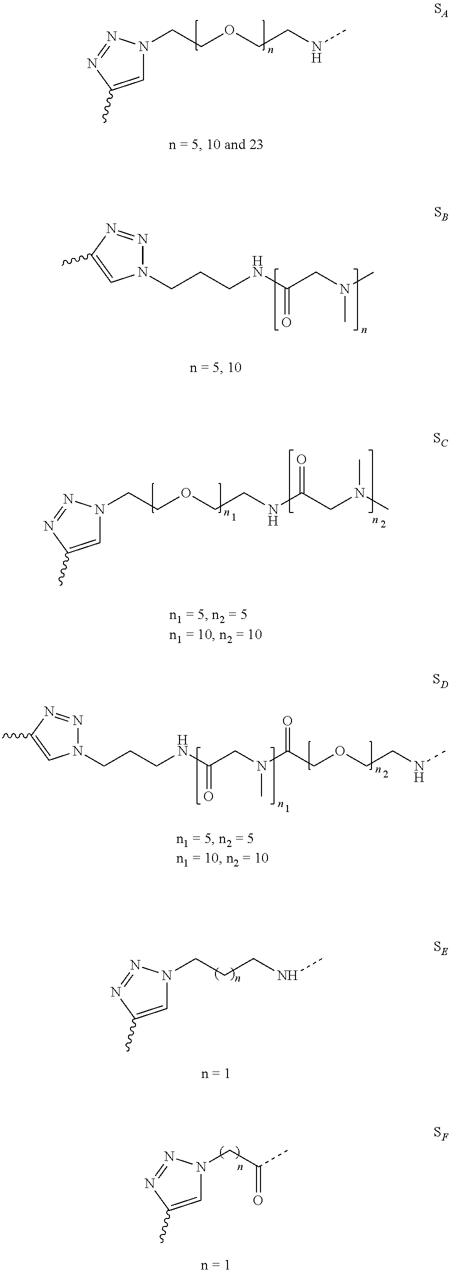

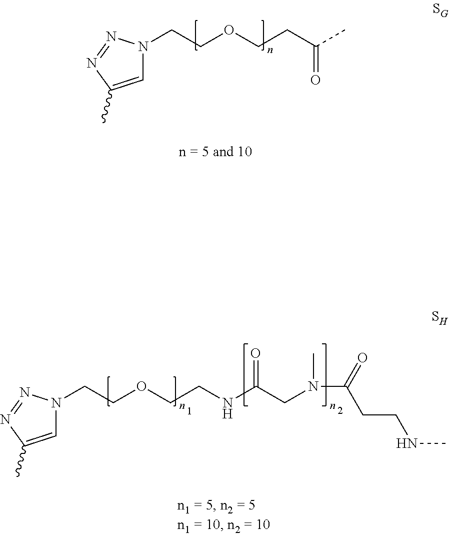

wherein “-----” represents the point of attachment to the CHM group; and
“ [symbol] ” represents the point of attachment to the Bicycle or Modifier group.

In a yet further embodiment, the spacer (S₁ and S₂) is S_{A} and n is 5, 10 or 23, such as 10 or 23.

It will be appreciated that the bicyclic peptide ligand may be attached to the spacer via a number of means. In one embodiment, the bicyclic peptide ligand is conjugated to one half of a binding pair and said other half of said binding pair links each of the bicyclic peptides to the spacer.

In one embodiment, said binding pair comprises biotin and streptavidin. Thus, each bicyclic peptide ligand is conjugated to biotin and linked to the spacer via streptavidin.

In one embodiment, one or both of said spacers (S₁ and S₂) are absent, i.e. there is a direct bond which links either Bicycle₁ and CHM and/or Bicycle₂ and CHM and/or CHM and Modifier.

In a further embodiment, both of said spacers (S₁ and S₂) are absent. In a yet further embodiment, n represents 2, CHM is a motif of formula (D) and both of said spacers (S₁ and S₂) are absent.

### Bicyclic Peptides

It will be appreciated that the multimeric binding complexes herein will comprise a plurality of monomeric bicyclic peptides.

### CD137 Bicyclic Peptide Monomers

In one embodiment, each of said peptide ligands (i.e. monomers) is specific for CD137, such as human CD137.

In one embodiment, said loop sequences comprise 6 amino acids.

In a further embodiment, said loop sequences comprise three cysteine residues separated by two loop sequences both of which consist of 6 amino acids.

In a yet further embodiment, said peptide ligand comprises a core amino acid sequence selected from:

(SEQ ID NO: 1)
CᵢIEEGQYCᵢᵢFADPY[Nle]Cᵢᵢᵢ;
(SEQ ID NO: 2)
CᵢIEEGQYCᵢᵢFADPYMCᵢᵢᵢ;
(SEQ ID NO: 3)
CᵢIEE[dK(PYA)]QYCᵢᵢFADPY[Nle]Cᵢᵢᵢ;
(SEQ ID NO: 4)
[dCᵢ][dI][dE][dE]K[dQ][dY][dCᵢᵢ][dF][dA][dD][dP]
[dY][dNle][dCᵢᵢᵢ];
(SEQ ID NO: 5)
[dCᵢ][dI][dE][dE]K(PYA)[dQ][dY][dCᵢᵢ][dF][dA][dD]
[dP][dY][dNle][dCᵢᵢᵢ];
(SEQ ID NO: 6)
Cᵢ[tBuAla]PK(PYA)[dA]PYCᵢᵢFADPY[Nle]Cᵢᵢᵢ;
and
(SEQ ID NO: 7)
Cᵢ[tBuAla]PE[D-Lys(PYA)]PYCᵢᵢFADPY[Nle]Cᵢᵢᵢ;

wherein Cᵢ, Cᵢᵢ and Cᵢᵢᵢ represent first, second and third cysteine residues, respectively, Nle represents norleucine, PYA represents propargyl-acid and tBuAla represents t-butyl-alanine, or a pharmaceutically acceptable salt thereof.

In a yet further embodiment, said peptide ligand comprises N and C terminal additions and comprises an amino acid sequence selected from:
Ac-A-(SEQ ID NO: 1)-[Dap(PYA)]-CONH₂ (hereinafter referred to as BCY7741);
Ac-A-(SEQ ID NO: 1)-[Dap(Lys(PYA))]-CONH₂ (hereinafter referred to as BCY12799);
Ac-(SEQ ID NO: 2)-A-Pra-CONH₂ (hereinafter referred to as BCY7077);
Ac-A-(SEQ ID NO: 3)-A-CONH₂ (hereinafter referred to as BCY7744);
Ac-A-(SEQ ID NO: 3)-K—CONH₂ (hereinafter referred to as BCY11613);
Ac-[dA]-(SEQ ID NO: 4)-[dA]-CONH₂ (hereinafter referred to as BCY11506);
Ac-[dA]-(SEQ ID NO: 5)-[dK]-CONH₂ (hereinafter referred to as BCY12144);
Ac-(SEQ ID NO: 6)-A-CONH₂ (hereinafter referred to as BCY8927);
Ac-(SEQ ID NO: 6)-K—CONH₂ (hereinafter referred to as BCY12357);
Ac-(SEQ ID NO: 7)-A (herein referred to as BCY8928); and
Ac-(SEQ ID NO: 7)-K (herein referred to as BCY13389);
   wherein Dap represents diaminopropionic acid, PYA represents propargyl-acid and Pra represents propargylglycine, or a pharmaceutically acceptable salt thereof.

### Nectin-4 Bicyclic Peptide Monomers

In an alternative embodiment, at least one of said peptide ligands (i.e monomers) is specific for CD137 (i.e. is selected from one or more (such as two) of the above mentioned CD137 bicyclic peptide monomers) and at least one (such as one) of said peptide ligands (i.e. monomers) is specific for Nectin-4.

In one embodiment, said loop sequences comprise 3 or 8 amino acids.

In a further embodiment, said loop sequences comprise three cysteine residues separated by two loop sequences one of which consists of 3 amino acids and the other of which consists of 8 amino acids.

In a yet further embodiment, said peptide ligand comprises a core amino acid sequence which is:
CᵢP[1Nal][dD]CᵢᵢM[HArg]DWSTP[HyP]WCᵢᵢᵢ (SEQ ID NO: 8; herein referred to as BCY8116);
   wherein Cᵢ, Cᵢᵢ and Cᵢᵢᵢ represent first, second and third cysteine residues, respectively, 1Nal represents 1-naphthylalanine, HArg represents homoarginine and HyP represents hydroxyproline, or a pharmaceutically acceptable salt thereof.

### EphA2 Bicyclic Peptide Monomers

In an alternative embodiment, at least one of said peptide ligands (i.e monomers) is specific for CD137 (i.e. is selected from one or more (such as two) of the above mentioned CD137 bicyclic peptide monomers) and at least one (such as one) of said peptide ligands (i.e. monomers) is specific for EphA2.

In one embodiment, said loop sequences comprise 6 amino acids.

In a further embodiment, said loop sequences comprise three cysteine residues separated by two loop sequences both of which consist of 6 amino acids.

In a yet further embodiment, said peptide ligand comprises a core amino acid sequence selected from:

(SEQ ID NO: 9)
Cᵢ[HyP]LVNPLCᵢᵢLHP[dD]W[HArg]Cᵢᵢᵢ;
(SEQ ID NO: 10)
Cᵢ[HyP]LVNPLCᵢᵢLEP[d1Nal]WTCᵢᵢᵢ;

wherein Cᵢ, Cᵢᵢ and Cᵢᵢᵢ represent first, second and third cysteine residues, respectively, 1 Nal represents 1-naphthylalanine, HArg represents homoarginine and HyP represents hydroxyproline, or a pharmaceutically acceptable salt thereof.

In a yet further embodiment, said peptide ligand comprises N and C terminal additions and comprises an amino acid sequence selected from:
A-[HArg]-D-(SEQ ID NO: 9) (herein referred to as BCY9594); and
A-[HArg]-D-(SEQ ID NO: 10) (herein referred to as BCY13118);
   wherein HArg represents homoarginine or a pharmaceutically acceptable salt thereof.

In one embodiment, the molecular scaffold is 1,1′,1″-(1,3,5-triazinane-1,3,5-triyl)triprop-2-en-1-one (TATA).

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, N.Y.; Ausubel et al., Short Protocols in Molecular Biology (1999) 4^{th} ed., John Wiley & Sons, Inc.), which are incorporated herein by reference.

### Numbering

When referring to amino acid residue positions within peptides of the invention, cysteine residues (Cᵢ, Cᵢᵢ and Cᵢᵢᵢ) are omitted from the numbering as they are invariant, therefore, the numbering of amino acid residues within the peptides of the invention is referred to as below:

(SEQ ID NO: 1)
Cᵢ-I₁-E₂-E₃-G₄-Q₅-Y₆-Cᵢᵢ-F₇-A₈-D₉-P₁₀-Y₁₁-Nle₁₂-Cᵢᵢᵢ.

For the purpose of this description, all bicyclic peptides are assumed to be cyclised with TBMB (1,3,5-tris(bromomethyl)benzene) or 1,1′,1″-(1,3,5-triazinane-1,3,5-triyl)triprop-2-en-1-one (TATA) and yielding a tri-substituted structure. Cyclisation with TBMB and TATA occurs on Cᵢ, Cᵢᵢ, and Cᵢᵢᵢ.

### Molecular Format

N- or C-terminal extensions to the bicycle core sequence are added to the left or right side of the sequence, separated by a hyphen. For example, an N-terminal βAla-Sar₁₀-Ala tail would be denoted as:

(SEQ ID NO: X)
βAla-Sar₁₀-A-.

### Inversed Peptide Sequences

In light of the disclosure in Nair et al (2003) J Immunol 170(3), 1362-1373, it is envisaged that the peptide sequences disclosed herein would also find utility in their retro-inverso form. For example, the sequence is reversed (i.e. N-terminus becomes C-terminus and vice versa) and their stereochemistry is likewise also reversed (i.e. D-amino acids become L-amino acids and vice versa).

### Peptide Ligands

A peptide ligand, as referred to herein, refers to a peptide covalently bound to a molecular scaffold. Typically, such peptides comprise two or more reactive groups (i.e. cysteine residues) which are capable of forming covalent bonds to the scaffold, and a sequence subtended between said reactive groups which is referred to as the loop sequence, since it forms a loop when the peptide is bound to the scaffold. In the present case, the peptides comprise at least three cysteine residues (referred to herein as Cᵢ, Cᵢᵢ and Cᵢᵢᵢ), and form at least two loops on the scaffold.

### Multimeric Binding Complexes

In one embodiment, the modified multimeric binding complex comprises a binding complex described in the following Table 1:

TABLE 1
Exemplified Modified Multimeric Binding Complexes of the Invention

| Multimeric | | | | | | | | |
|---|---|---|---|---|---|---|---|---|
| Number | Formula | CHM | Bicycle₁ | S₁ | m/n | Bicycle₂ | S₂ | Modifier |
| BCY9931 | (I) | A | BCY12799 | S_{A}: n = | m = 3 | BCY12799 | S_{A}: n = | Alexa |
| | | | | 23 | | | 23 | Fluor ™ |
| | | | | | | | | 488- |
| | | | | | | | | COOH |
| BCY9932 | (II) | B | BCY7741 | S_{A}: n = | n = 3 | — | No | Alexa |
| | | | | 10 | | | Spacer | Fluor ™ |
| | | | | | | | | 488- |
| | | | | | | | | COOH |
| BCY7340 | (II) | C | BCY7077 | S_{A}: n = | n = 2 | — | No | Fluorescein- |
| | | | | 23 | | | Spacer | COOH |
| BCY11467 | (II) | A | BCY7744 | S_{A}: n = | n = 3 | — | S_{A}: n = | 1X |
| | | | | 10 | | | 10 | Palmitoyl- |
| | | | | | | | | Pra-COON |
| BCY11554 | (II) | A | BCY7744 | S_{A}: n = | n = 3 | — | S_{A}: n = | 1 X |
| | | | | 10 | | | 10 | Biotin- |
| | | | | | | | | Peg4- |
| | | | | | | | | alkyne |
| BCY11856 | (II) | A | BCY11506 | S_{A}: n = | n = 3 | — | S_{A}: n = | 1 X |
| | | | | 10 | | | 10 | Alexa |
| | | | | | | | | Fluor ™ |
| | | | | | | | | 488 |
| | | | | | | | | Alkyne |
| | | | | | | | | (Alexa |
| | | | | | | | | Fluor ™ |
| | | | | | | | | 488 5- |
| | | | | | | | | Carboxamido- |
| | | | | | | | | (Propargyl) |
| BCY11862 | (I) | A | BCY7744 | S_{A}: n = | m = 3 | BCY11613 | S_{A}: n = | Biotin- |
| | | | | 23 | | | 23 | Peg12- |
| | | | | | | | | COOH |
| BCY12239 | (I) | A | BCY7744 | S_{A}: n = | m = 3 | BCY11613 | S_{A}: n = | Alexa |
| | | | | 23 | | | 23 | Fluor ™ |
| | | | | | | | | 488- |
| | | | | | | | | COOH |
| BCY12240 | (I) | A | BCY7744 | S_{A}: n = | m = 3 | BCY11613 | S_{A}: n = | Cyanine |
| | | | | 23 | | | 23 | 5-COOH |
| BCY12380 | (I) | A | BCY7744 | S_{A}: n = | m = 3 | BCY11613 | S_{A}: n = | BODIPY ™ FL- |
| | | | | 23 | | | 23 | COOH |
| BCY12374 | (I) | A | BCY11506 | S_{A}: n = | m = 3 | BCY12144 | S_{A}: n = | Biotin- |
| | | | | 23 | | | 23 | Peg12- |
| | | | | | | | | COOH |
| BCY12657 | (I) | A | BCY8927 | S_{A}: n = | m = 3 | BCY12357 | S_{A}: n = | Biotin- |
| | | | | 23 | | | 23 | Peg12- |
| | | | | | | | | COOH |
| BCY12660 | (I) | A | BCY8927 | S_{A}: n = | m = 3 | BCY12357 | S_{A}: n = | BODIPY ™ |
| | | | | 23 | | | 23 | FL- |
| | | | | | | | | COOH |
| BCY12658 | (I) | A | BCY8927 | S_{A}: n = | m = 3 | BCY12357 | S_{A}: n = | Alexa |
| | | | | 23 | | | 23 | Fluor ™ |
| | | | | | | | | 488- |
| | | | | | | | | COOH |
| BCY12659 | (I) | A | BCY8927 | S_{A}: n = | m = 3 | BCY12357 | S_{A}: n = | Cyanine |
| | | | | 23 | | | 23 | 5-COOH |
| BCY15416 | (II) | D | BCY8928 | No | n = 2 | — | No | Alexa |
| | | | | spacer | | | spacer | Fluor ™ |
| | | | | | | | | 488- |
| | | | | | | | | COOH |
| BCY15985 | (II) | D | BCY8928 | No | n = 2 | — | No | Biotin- |
| | | | | spacer | | | spacer | Peg12- |
| | | | | | | | | COOH |

In one embodiment, the modified multimeric binding complex comprises a binding complex which is other than BCY12374. BCY12374 represents a non-binding control.

TABLE 2
Composition of Nectin-4 targeted modified multimer complexes

| Complex | Nectin-4 | Attachment | | CD137 | Attachment | |
|---|---|---|---|---|---|---|
| No. | BCY No. | Point | Linker | BCY No. | Point | Modifier |
| BCY13582 | BCY8116 | N-terminus | N-(acid-PEG₃)-N- | BCY8928 | dLys(PYA)4 | Biotin- |
| | | | bis(PEG₃-azide) | BCY13389 | dLys(PYA)4 | Peg12 |
| BCY13583 | BCY8116 | N-terminus | N-(acid-PEG₃)-N- | BCY8928 | dLys(PYA)4 | Alexa |
| | | | bis(PEG₃-azide) | BCY13389 | dLys(PYA)4 | Fluor 488 |
| BCY13628 | BCY8116 | N-terminus | N-(acid-PEG₃)-N- | BCY8928 | dLys(PYA)4 | Cyanine 5 |
| | | | bis(PEG₃-azide) | BCY13389 | dLys(PYA)4 | |

TABLE 3
Composition of EphA2 tarqeted modified multimer complexes

| Complex | EphA2 | Attachment | | CD137 BCY | Attachment | |
|---|---|---|---|---|---|---|
| No. | BCY No. | Point | Linker | No. | Point | Modifier |
| BCY14417 | BCY13118 | N- | N-(acid-PEG₃)-N- | BCY8928 | dLys(PYA)4 | Peg12-Biotin |
| | | terminus | bis(PEG₃-azide) | BCY13389 | dLys(PYA)4 | |
| BCY14418 | BCY13118 | N- | N-(acid-PEG₃)-N- | BCY8928 | dLys(PYA)4 | Alexa |
| | | terminus | bis(PEG₃-azide) | BCY13389 | dLys(PYA)4 | Fluor ® 488 |
| BCY14415 | BCY9594 | N- | N-(acid-PEG3)-N- | BCY8928 | dLys (PYA)4 | Peg12-Biotin |
| | | terminus | bis(PEG3-azide) | BCY13389 | dLys (PYA)4 | |
| BCY14416 | BCY9594 | N- | N-(acid-PEG3)-N- | BCY8928 | dLys (PYA)4 | Alexa |
| | | terminus | bis(PEG3-azide) | BCY13389 | dLys (PYA)4 | Fluor ® 488 |

### Pharmaceutically Acceptable Salts

It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic, or has a functional group which may be anionic (e.g., —COOH may be —COO⁻), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the peptides of the invention contain an amine function, these may form quaternary ammonium salts, for example by reaction with an alkylating agent according to methods well known to the skilled person. Such quaternary ammonium compounds are within the scope of the peptides of the invention.

### Isotopic Variations

The present invention includes all pharmaceutically acceptable (radio)isotope-labelled peptide ligands of the invention, wherein one or more atoms are replaced by atoms having the same atomic number, but an atomic mass or mass number different from the atomic mass or mass number usually found in nature, and peptide ligands of the invention, wherein metal chelating groups are attached (termed “effector”) that are capable of holding relevant (radio)isotopes, and peptide ligands of the invention, wherein certain functional groups are covalently replaced with relevant (radio)isotopes or isotopically labelled functional groups.

Examples of isotopes suitable for inclusion in the peptide ligands of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I and ¹³¹I, nitrogen, such as ¹³N and ¹⁵N, oxygen, such as ¹⁵O, ¹⁷O and ¹⁸O, phosphorus, such as ³²P, sulphur, such as ³⁵S, copper, such as ⁶⁴Cu, gallium, such as ⁶⁷Ga or ⁶⁸Ga, yttrium, such as ⁹⁰Y and lutetium, such as ¹⁷⁷Lu, and Bismuth, such as ²¹³Bi.

Certain isotopically-labelled peptide ligands of the invention, for example, those incorporating a radioactive isotope, are useful in drug and/or substrate tissue distribution studies, and to clinically assess the presence and/or absence of the CD137 target on diseased tissues. The peptide ligands of the invention can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labelled compounds of peptide ligands of the invention can generally be prepared by conventional techniques known to those skilled in the art or by processes analogous to those described in the accompanying Examples using an appropriate isotopically-labeled reagent in place of the non-labeled reagent previously employed.

### Molecular Scaffold

Molecular scaffolds are described in, for example, WO 2009/098450 and references cited therein, particularly WO 2004/077062 and WO 2006/078161.

As noted in the foregoing documents, the molecular scaffold may be a small molecule, such as a small organic molecule.

In one embodiment the molecular scaffold may be a macromolecule. In one embodiment the molecular scaffold is a macromolecule composed of amino acids, nucleotides or carbohydrates.

In one embodiment the molecular scaffold comprises reactive groups that are capable of reacting with functional group(s) of the polypeptide to form covalent bonds.

The molecular scaffold may comprise chemical groups which form the linkage with a peptide, such as amines, thiols, alcohols, ketones, aldehydes, nitriles, carboxylic acids, esters, alkenes, alkynes, azides, anhydrides, succinimides, maleimides, alkyl halides and acyl halides.

In one embodiment, the molecular scaffold may comprise or may consist of hexahydro-1,3,5-triazine, especially 1,3,5-triacryloylhexahydro-1,3,5-triazine (‘TATA’), or a derivative thereof.

In one embodiment, the molecular scaffold is 2,4,6-tris(bromomethyl)mesitylene. This molecule is similar to 1,3,5-tris(bromomethyl)benzene (TBMB) but contains three additional methyl groups attached to the benzene ring. This has the advantage that the additional methyl groups may form further contacts with the polypeptide and hence add additional structural constraint.

The molecular scaffold of the invention contains chemical groups that allow functional groups of the polypeptide of the encoded library of the invention to form covalent links with the molecular scaffold. Said chemical groups are selected from a wide range of functionalities including amines, thiols, alcohols, ketones, aldehydes, nitriles, carboxylic acids, esters, alkenes, alkynes, anhydrides, succinimides, maleimides, azides, alkyl halides and acyl halides.

Scaffold reactive groups that could be used on the molecular scaffold to react with thiol groups of cysteines are alkyl halides (or also named halogenoalkanes or haloalkanes).

Examples include bromomethylbenzene or iodoacetamide. Other scaffold reactive groups that are used to selectively couple compounds to cysteines in proteins are maleimides, αβ unsaturated carbonyl containing compounds and α-halomethylcarbonyl containing compounds. Examples of maleimides which may be used as molecular scaffolds in the invention include: tris-(2-maleimidoethyl)amine, tris-(2-maleimidoethyl)benzene, tris-(maleimido)benzene. An example of an αβ unsaturated carbonyl containing compound is 1,1′,1″-(1,3,5-triazinane-1,3,5-triyl)triprop-2-en-1-one (TATA) (Angewandte Chemie, International Edition (2014), 53(6), 1602-1606). An example of an α-halomethylcarbonyl containing compound is N,N′,N″-(benzene-1,3,5-triyl)tris(2-bromoacetamide). Selenocysteine is also a natural amino acid which has a similar reactivity to cysteine and can be used for the same reactions. Thus, wherever cysteine is mentioned, it is typically acceptable to substitute selenocysteine unless the context suggests otherwise.

### Effector and Functional Groups

According to a further aspect of the invention, there is provided a drug conjugate comprising a multimeric binding complex as defined herein conjugated to one or more effector and/or functional groups.

Effector and/or functional groups can be attached, for example, to the N and/or C termini of the polypeptide, to an amino acid within the polypeptide, or to the molecular scaffold.

Appropriate effector groups include antibodies and parts or fragments thereof. For instance, an effector group can include an antibody light chain constant region (CL), an antibody CH1 heavy chain domain, an antibody CH2 heavy chain domain, an antibody CH3 heavy chain domain, or any combination thereof, in addition to the one or more constant region domains. An effector group may also comprise a hinge region of an antibody (such a region normally being found between the CH1 and CH2 domains of an IgG molecule).

In a further embodiment of this aspect of the invention, an effector group according to the present invention is an Fc region of an IgG molecule. Advantageously, a peptide ligand-effector group according to the present invention comprises or consists of a peptide ligand Fc fusion having a tβ half-life of a day or more, two days or more, 3 days or more, 4 days or more, 5 days or more, 6 days or more or 7 days or more. Most advantageously, the peptide ligand according to the present invention comprises or consists of a peptide ligand Fc fusion having a half-life of a day or more.

Functional groups include, in general, binding groups, drugs, reactive groups for the attachment of other entities, functional groups which aid uptake of the macrocyclic peptides into cells, and the like.

The ability of peptides to penetrate into cells will allow peptides against intracellular targets to be effective. Targets that can be accessed by peptides with the ability to penetrate into cells include transcription factors, intracellular signalling molecules such as tyrosine kinases and molecules involved in the apoptotic pathway. Functional groups which enable the penetration of cells include peptides or chemical groups which have been added either to the peptide or the molecular scaffold. Peptides such as those derived from such as VP22, HIV-Tat, a homeobox protein of Drosophila (Antennapedia), e.g. as described in Chen and Harrison, Biochemical Society Transactions (2007) Volume 35, part 4, p 821; Gupta et al. in Advanced Drug Discovery Reviews (2004) Volume 57 9637. Examples of short peptides which have been shown to be efficient at translocation through plasma membranes include the 16 amino acid penetratin peptide from Drosophila Antennapedia protein (Derossi et al (1994) J Biol. Chem. Volume 269 p 10444), the 18 amino acid ‘model amphipathic peptide’ (Oehlke et al (1998) Biochim Biophys Acts Volume 1414 p 127) and arginine rich regions of the HIV TAT protein. Non peptidic approaches include the use of small molecule mimics or SMOCs that can be easily attached to biomolecules (Okuyama et al (2007) Nature Methods Volume 4 p 153). Other chemical strategies to add guanidinium groups to molecules also enhance cell penetration (Elson-Scwab et al (2007) J Biol Chem Volume 282 p 13585). Small molecular weight molecules such as steroids may be added to the molecular scaffold to enhance uptake into cells.

One class of functional groups which may be attached to peptide ligands includes antibodies and binding fragments thereof, such as Fab, Fv or single domain fragments. In particular, antibodies which bind to proteins capable of increasing the half-life of the peptide ligand in vivo may be used.

In one embodiment, a peptide ligand-effector group according to the invention has a tβ half-life selected from the group consisting of: 12 hours or more, 24 hours or more, 2 days or more, 3 days or more, 4 days or more, 5 days or more, 6 days or more, 7 days or more, 8 days or more, 9 days or more, 10 days or more, 11 days or more, 12 days or more, 13 days or more, 14 days or more, 15 days or more or 20 days or more. Advantageously a peptide ligand-effector group or composition according to the invention will have a tβ half-life in the range 12 to 60 hours. In a further embodiment, it will have a tβ half-life of a day or more. In a further embodiment still, it will be in the range 12 to 26 hours.

In one particular embodiment of the invention, the functional group is selected from a metal chelator, which is suitable for complexing metal radioisotopes of medicinal relevance.

Possible effector groups also include enzymes, for instance such as carboxypeptidase G2 for use in enzyme/prodrug therapy, where the peptide ligand replaces antibodies in ADEPT.

In one embodiment, the multimeric binding complexes of the invention contain a cleavable bond, such as a disulphide bond or a protease sensitive bond. Without being bound by theory it is believed that such a cleavable moiety deactivates the complex until it reaches the tumour microenvironment. The benefit of this embodiment provides for the complex to be reduced in size following binding to the target. In a further embodiment, the groups adjacent to the disulphide bond are modified to control the hindrance of the disulphide bond, and by this the rate of cleavage and concomitant release of the binding agent.

Published work established the potential for modifying the susceptibility of the disulphide bond to reduction by introducing steric hindrance on either side of the disulphide bond (Kellogg et al (2011) Bioconjugate Chemistry, 22, 717). A greater degree of steric hindrance reduces the rate of reduction by intracellular glutathione and also extracellular (systemic) reducing agents, consequentially reducing the ease by which toxin is released, both inside and outside the cell. Thus, selection of the optimum in disulphide stability in the circulation (which minimises undesirable side effects of the toxin) versus efficient release in the intracellular milieu (which maximises the therapeutic effect) can be achieved by careful selection of the degree of hindrance on either side of the disulphide bond.

The hindrance on either side of the disulphide bond is modulated through introducing one or more methyl groups on the targeting entity (here, the bicyclic peptide).

### Synthesis

The peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al (supra).

Thus, the invention also relates to the manufacture of polypeptides or conjugates selected as set out herein, wherein the manufacture comprises optional further steps as explained below. In one embodiment, these steps are carried out on the end product polypeptide/conjugate made by chemical synthesis.

Optionally amino acid residues in the polypeptide of interest may be substituted when manufacturing a conjugate or complex.

Peptides can also be extended, to incorporate for example another loop and therefore introduce multiple specificities.

To extend the peptide, it may simply be extended chemically at its N-terminus or C-terminus or within the loops using orthogonally protected lysines (and analogues) using standard solid phase or solution phase chemistry. Standard (bio)conjugation techniques may be used to introduce an activated or activatable N- or C-terminus. Alternatively additions may be made by fragment condensation or native chemical ligation e.g. as described in (Dawson et al. 1994. Synthesis of Proteins by Native Chemical Ligation. Science 266:776-779), or by enzymes, for example using subtiligase as described in (Chang et al Proc Natl Acad Sci USA. 1994 Dec. 20; 91(26):12544-8 or in Hikari et al Bioorganic & Medicinal Chemistry Letters Volume 18, Issue 22, 15 Nov. 2008, Pages 6000-6003).

Alternatively, the peptides may be extended or modified by further conjugation through disulphide bonds. This has the additional advantage of allowing the first and second peptide to dissociate from each other once within the reducing environment of the cell. In this case, the molecular scaffold (e.g. TATA) could be added during the chemical synthesis of the first peptide so as to react with the three cysteine groups; a further cysteine or thiol could then be appended to the N or C-terminus of the first peptide, so that this cysteine or thiol only reacted with a free cysteine or thiol of the second peptide, forming a disulphide-linked bicyclic peptide-peptide conjugate.

Similar techniques apply equally to the synthesis/coupling of two bicyclic and bispecific macrocycles, potentially creating a tetraspecific molecule.

Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

### Pharmaceutical Compositions

According to a further aspect of the invention, there is provided a pharmaceutical composition comprising a multimeric binding complex or a drug conjugate as defined herein in combination with one or more pharmaceutically acceptable excipients.

Generally, the present peptide ligands will be utilised in purified form together with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The peptide ligands of the present invention may be used as separately administered compositions or in conjunction with other agents. These can include antibodies, antibody fragments and various immunotherapeutic drugs, such as cyclosporine, methotrexate, adriamycin or cisplatinum and immunotoxins. Pharmaceutical compositions can include “cocktails” of various cytotoxic or other agents in conjunction with the protein ligands of the present invention, or even combinations of selected polypeptides according to the present invention having different specificities, such as polypeptides selected using different target ligands, whether or not they are pooled prior to administration.

The route of administration of pharmaceutical compositions according to the invention may be any of those commonly known to those of ordinary skill in the art. For therapy, the peptide ligands of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. Preferably, the pharmaceutical compositions according to the invention will be administered by inhalation. The dosage and frequency of administration will depend on the age, sex and condition of the patient, concurrent administration of other drugs, counterindications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that levels may have to be adjusted upward to compensate.

The compositions containing the present peptide ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected peptide ligand per kilogram of body weight, with doses of 0.05 to 2.0 mg/kg/dose being more commonly used. For prophylactic applications, compositions containing the present peptide ligands or cocktails thereof may also be administered in similar or slightly lower dosages.

A composition containing a peptide ligand according to the present invention may be utilised in prophylactic and therapeutic settings to aid in the alteration, inactivation, killing or removal of a select target cell population in a mammal. In addition, the peptide ligands described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

### Therapeutic Uses

The bicyclic peptides of the invention have specific utility as CD137 binding agents.

CD137 is a member of the tumour necrosis factor (TNF) receptor family. Its alternative names are tumour necrosis factor receptor superfamily member 9 (TNFRSF9), 4-IBB and induced by lymphocyte activation (ILA). CD137 can be expressed by activated T cells, but to a larger extent on CD8+ than on CD4+ T cells. In addition, CD137 expression is found on dendritic cells, follicular dendritic cells, natural killer cells, granulocytes and cells of blood vessel walls at sites of inflammation. One characterized activity of CD137 is its costimulatory activity for activated T cells. Crosslinking of CD137 enhances T cell proliferation, IL-2 secretion, survival and cytolytic activity. Further, it can enhance immune activity to eliminate tumours in mice.

CD137 is a T-cell costimulatory receptor induced on TCR activation (Nam et al., Curr. Cancer Drug Targets, 5:357-363 (2005); Waits et al., Annu. Rev, Immunol., 23:23-68 (2005)). In addition to its expression on activated CD4+ and CD8+ T cells, CD137 is also expressed on CD4+CD25+ regulatory T cells, natural killer (NK) and NK-T cells, monocytes, neutrophils, and dendritic cells. Its natural ligand, CD137L, has been described on antigen-presenting cells including B cells, monocyte/macrophages, and dendritic cells (Watts et al. Annu. Rev. Immunol, 23:23-68 (2005)). On interaction with its ligand, CD137 leads to increased TCR-induced T-cell proliferation, cytokine production, functional maturation, and prolonged CD8+ T-cell survival (Nam et al, Curr. Cancer Drug Targets, 5:357-363 (2005), Watts et al., Annu. Rev. Immunol, 23:23-68 (2005)).

Signalling through CD137 by either CD137L or agonistic monoclonal antibodies (mAbs) against CD137 leads to increased TCR-induced T cell proliferation, cytokine production and functional maturation, and prolonged CD8+ T cell survival. These effects result from: (1) the activation of the NF-κB, c-Jun NH₂-terminal kinase/stress-activated protein kinase (JNK/SAPK), and p38 mitogen-activated protein kinase (MAPK) signalling pathways, and (2) the control of anti-apoptotic and cell cycle-related gene expression.

Experiments performed in both CD137 and CD137L-deficient mice have additionally demonstrated the importance of CD137 costimulation in the generation of a fully competent T cell response.

IL-2 and IL-15 activated NK cells express CD137, and ligation of CD137 by agonistic mAbs stimulates NK cell proliferation and IFN-γ secretion, but not their cytolytic activity.

Furthermore, CD137-stimulated NK cells promote the expansion of activated T cells in vitro.

In accordance with their costimulatory function, agonist mAbs against CD137 have been shown to promote rejection of cardiac and skin allografts, eradicate established tumours, broaden primary antiviral CD8+ T cell responses, and increase T cell cytolytic potential. These studies support the view that CD137 signalling promotes T cell function which may enhance immunity against tumours and infection.

Polypeptide ligands selected according to the method of the present invention may be employed in in vivo therapeutic and prophylactic applications, in vitro and in vivo diagnostic applications, in vitro assay and reagent applications, and the like. Ligands having selected levels of specificity are useful in applications which involve testing in non-human animals, where cross-reactivity is desirable, or in diagnostic applications, where cross-reactivity with homologues or paralogues needs to be carefully controlled. In some applications, such as vaccine applications, the ability to elicit an immune response to predetermined ranges of antigens can be exploited to tailor a vaccine to specific diseases and pathogens.

Substantially pure peptide ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

According to a further aspect of the invention, there is provided a multimeric binding complex or a drug conjugate as defined herein, for use in preventing, suppressing or treating a disease or disorder mediated by CD137.

According to a further aspect of the invention, there is provided a method of preventing, suppressing or treating a disease or disorder mediated by CD137, which comprises administering to a patient in need thereof an effector group and drug conjugate of the multimeric binding complex as defined herein.

In one embodiment, the CD137 is mammalian CD137. In a further embodiment, the mammalian CD137 is human CD137 (hCD137).

In one embodiment, the disease or disorder mediated by CD137 is selected from cancer, infection and inflammation. In a further embodiment, the disorder or disease mediated by CD137 is selected from cancer.

Examples of cancers (and their benign counterparts) which may be treated (or inhibited) include, but are not limited to tumours of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the oesophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukaemias, lymphomas) and premalignant haematological disorders and disorders of borderline malignancy including haematological malignancies and related conditions of lymphoid lineage (for example acute lymphocytic leukaemia [ALL], chronic lymphocytic leukaemia [CLL], B-cell lymphomas such as diffuse large B-cell lymphoma [DLBCL], follicular lymphoma, Burkitt's lymphoma, mantle cell lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenousleukemia [AML], chronic myelogenousleukemia [CML], chronic myelomonocyticleukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocyticleukaemia); tumours of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumours, benign and malignant histiocytomas, and dermatofibrosarcomaprotuberans; tumours of the central or peripheral nervous system (for example astrocytomas, gliomas and glioblastomas, meningiomas, ependymomas, pineal tumours and schwannomas); endocrine tumours (for example pituitary tumours, adrenal tumours, islet cell tumours, parathyroid tumours, carcinoid tumours and medullary carcinoma of the thyroid); ocular and adnexal tumours (for example retinoblastoma); germ cell and trophoblastic tumours (for example teratomas, seminomas, dysgerminomas, hydatidiform moles and choriocarcinomas); and paediatric and embryonal tumours (for example medulloblastoma, neuroblastoma, Wilms tumour, and primitive neuroectodermal tumours); or syndromes, congenital or otherwise, which leave the patient susceptible to malignancy (for example Xeroderma Pigmentosum).

In a further embodiment, the cancer is selected from a hematopoietic malignancy such as selected from: non-Hodgkin's lymphoma (NHL), Burkitt's lymphoma (BL), multiple myeloma (MM), B chronic lymphocytic leukaemia (B-CLL), B and T acute lymphocytic leukaemia (ALL), T cell lymphoma (TCL), acute myeloid leukaemia (AML), hairy cell leukaemia (HCL), Hodgkin's Lymphoma (HL), and chronic myeloid leukaemia (CML).

References herein to the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the peptide ligands in protecting against or treating the disease are available. The use of animal model systems is facilitated by the present invention, which allows the development of polypeptide ligands which can cross react with human and animal targets, to allow the use of animal models.

The invention is further described below with reference to the following examples.

### EXAMPLES

### Materials and Methods

### Peptide Synthesis

Peptide synthesis was based on Fmoc chemistry, using a Symphony peptide synthesiser manufactured by Peptide Instruments and a Syro II synthesiser by MultiSynTech. Standard Fmoc-amino acids were employed (Sigma, Merck), with appropriate side chain protecting groups: where applicable standard coupling conditions were used in each case, followed by deprotection using standard methodology. Peptides were purified using HPLC and following isolation they were modified with 1,3,5-Triacryloylhexahydro-1,3,5-triazine (TATA, Sigma). For this, linear peptide was diluted with 50:50 MeCN:H₂O up to ˜35 mL, ˜500 μL of 100 mM TATA in acetonitrile was added, and the reaction was initiated with 5 mL of 1 M NH₄HCO₃ in H₂O. The reaction was allowed to proceed for ˜30-60 min at RT, and lyophilised once the reaction had completed (judged by MALDI-MS). Once completed, 1 ml of 1M L-cysteine hydrochloride monohydrate (Sigma) in H₂O was added to the reaction for ˜60 min at RT to quench any excess TATA.

Following lyophilisation, the modified peptide was purified as above, while replacing the Luna C8 with a Gemini C18 column (Phenomenex), and changing the acid to 0.1% trifluoroacetic acid. Pure fractions containing the correct TATA-modified material were pooled, lyophilised and kept at −20° C. for storage.

All amino acids, unless noted otherwise, were used in the L-configurations.

### Multimer Synthesis

BCY9931

### Preparation of BCY9297

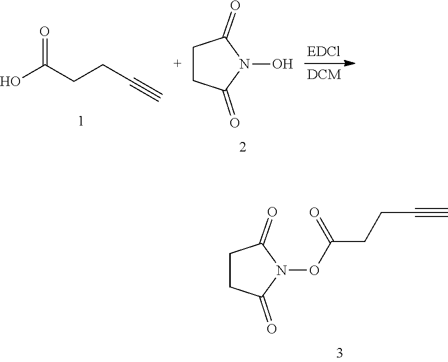

To a solution of compound 1 (2 g, 20.4 mmol) in DCM (50 ml-) was added EDCl (7.82 g, 40.8 mmol) and compound 2 (2.58 g, 22.43 mmol, 1.1 eq). The mixture was stirred at 25-30° C. for 1 hr. TLC (Eluent: Petroleum ether:Dichloromethane=0:1, Color Developing Reagent: Bromocresol green, Rf=0.44) indicated Reactant 1 was consumed completely and one new spot formed. The reaction was clean according to TLC. The reaction mixture was then concentrated under reduced pressure to give a residue, following by purification through flash silica gel chromatography (ISCO®; 40 g SepaFlash® Silica Flash Column, Eluent of 0-50% Ethyl acetate/Petroleum ether gradient @ 40 mL/min) to give compound 3 (2.8 g, 13.63 mmol, 66.8% yield) as a white solid.

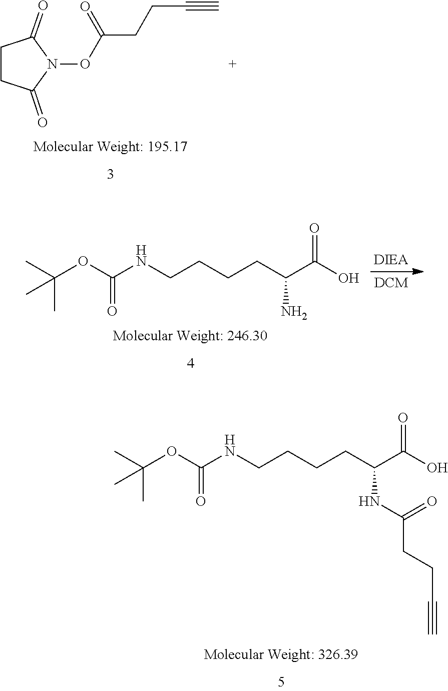 [SMILES: C#CCCC(=O)ON1C(=O)CCC1=O ; CC(C)(C)OC(=O)NCCCC[C@@H](N)C(=O)O ; C#CCCC(=O)N[C@H](CCCCNC(=O)OC(C)(C)C)C(=O)O]

To a solution of compound 4 (200 mg, 707.3 μmol, HCl) and compound 3 (151.8 mg, 778.0 μmol) in DCM (10 mL) was added DIEA (457.0 mg, 3.54 mmol, 616 μL). The mixture was stirred at 25-30° C. for 2 hrs. LC-MS showed desired mass was detected (observed m/z, [M+Na⁺], 349.2). The reaction mixture was concentrated under reduced pressure to remove solvent to give a residue. The residue was purified by prep-HPLC (neutral condition) to give compound 5 (200 mg, 551.49 μmol, 77.9% yield, 90% purity) was obtained as a white solid.

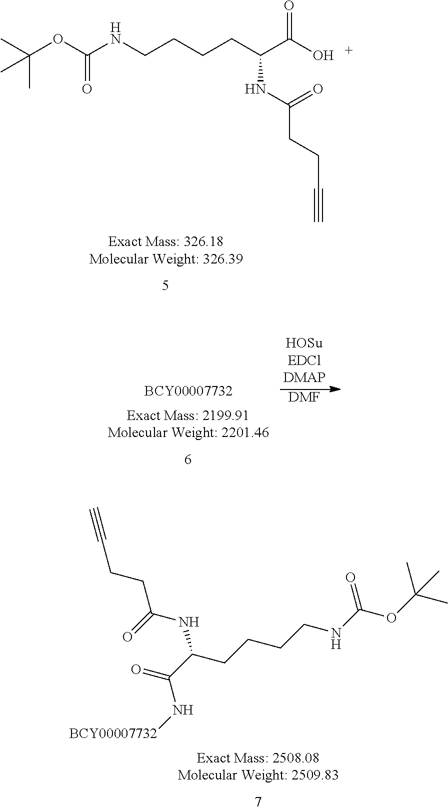

To a solution of compound 5 (20 mg, 61.28 μmol) in DMF (5 mL) was added HOSu (8.46 mg, 73.53 μmol), compound 6 (134.9 mg, 61.28 μmol) and EDCl (14.10 mg, 73.53 μmol), DMAP (2.25 mg, 18.38 μmol). The mixture was stirred at 25° C. for 2 hrs. LC-MS showed desired mass was detected (Calculated MW: 2509.83, observed m/z [(M-Boc)/5+H⁺] 482.3). The residue was purified by prep-HPLC (neutral condition) to give compound 7 (95 mg, 37.85 μmol, 61.7% yield) was obtained as a white solid.

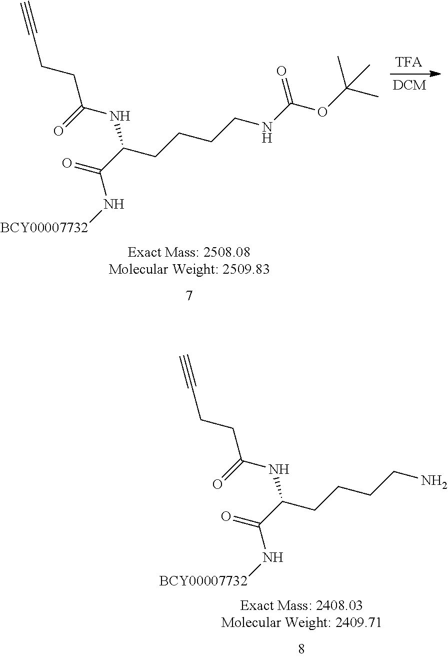

To a solution of compound 7 (95 mg, 37.85 μmol) in DCM (5 mL) was added TFA (4.32 mg, 37.85 μmol, 2.80 μL). The mixture was stirred at 25° C. for 0.5 hrs. LC-MS showed Reactant 1 was consumed completely and one main peak with desired m/z (Calculated MW: 2409.71, observed m/z: [M/2+H⁺] 1205.3, [M/3+H⁺] 803.8) was detected. The reaction mixture was concentrated under reduced pressure to remove solvent to give a residue. The residue was purified by prep-HPLC (neutral condition) to give compound 8 (60 mg, 24.90 μmol, 65.8% yield) was obtained as a white solid.

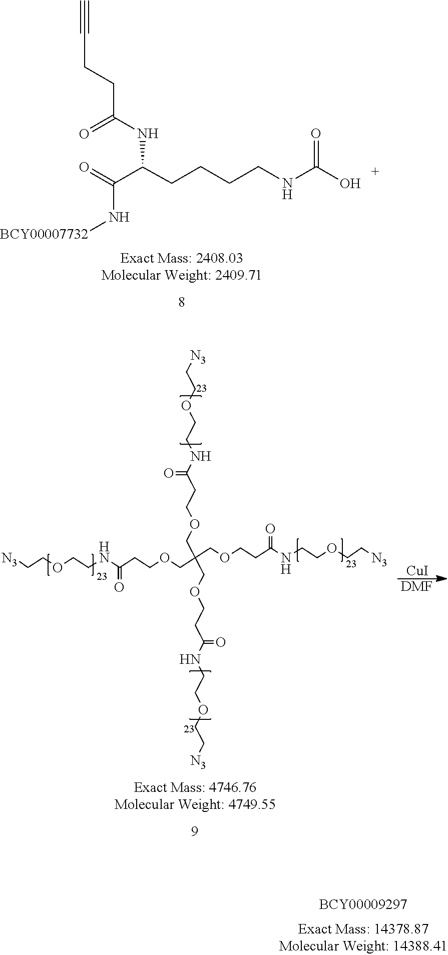

To a solution of compound 8 (60 mg, 24.9 μmol, 5.0 eq), compound 9 (23.6 mg, 5.0 μmol, 1.0 eq) in DMF (5 mL) was added CuI (23.71 mg, 124.5 μmol, 24.9 eq). The mixture was stirred at 25° C. for 16 hrs under N₂ atmosphere. LC-MS showed compound 8 was consumed completely and desired m/z was detected in HRMS (Calculated MW: 14388.41, observed m/z: [M/10+H] 1439.8430. The residue was purified by prep-HPLC (TFA condition) to give BCY9297-1 (6.3 mg, 95.30%) and BCY9297-2 (9.8 mg, 69.40%) was obtained as a white solid.

### Preparation of BCY9931

A solution of BCY9297 in DMF (370 μL, 1 mM, 3.7×10⁻⁷ mol) was added to a solution of Alexa Fluor 488 in DMF (370 μL, 1.5 mM, 5.6×10⁻⁷ mol). DIPEA (25 μL) was added and the mixture stirred at room temperature overnight. The solution was diluted up to 10 mL using 6M guanidinium hydrochloride and purified using semi preparative RP-HPLC, then lyophilized to give BCY9931 as a yellow solid (350 ug, 2.34×10⁻⁸ mol, 6.3%) with desired m/z (Calculated MW: 14906.01, observed m/z: [M/11+H⁺] 1356.03.

BCY9932

### Preparation of Compound 3

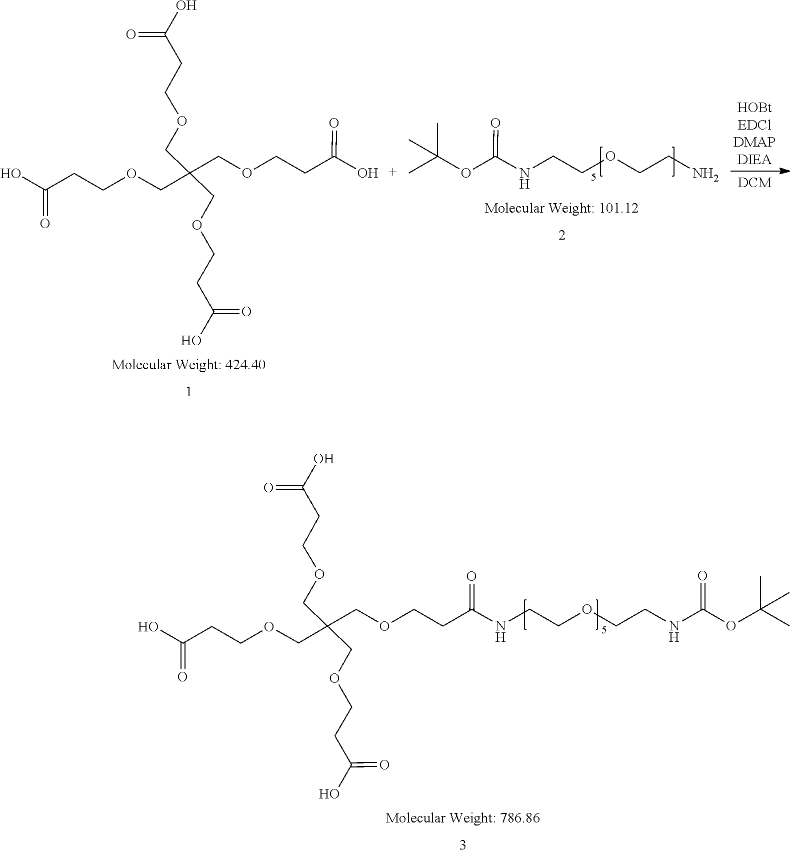

A mixture of compound 1 (200.0 mg, 471.3 μmol, 1.0 eq), compound 2 (179.3 mg, 471.3 μmol, 1.0 eq), EDCl (99.4 mg, 518.4 μmol, 1.1 eq), DMAP (63.3 mg, 518.4 μmol, 1.1 eq), HOBt (70.0 mg, 518.4 μmol, 1.1 eq) were dissolved in 10 mL DCM and mixed well, and then DIEA (304.5 mg, 2.36 mmol, 410.4 μL) was added. The mixture was stirred at 15-25° C. for 2 hrs under N₂ atmosphere. LC-MS showed Reactant 1 was consumed completely and one main peak with desired mass (calculated MW: 786.86, observed m/z: 787.2 [M+H⁺]) was detected. The reaction mixture was filtered and concentrated under reduced pressure to give a residue. The residue was purified by prep-HPLC (TFA condition) to give compound 3 (45 mg, 57.19 umol, 12.1% yield) was obtained as a yellow oil.

### Preparation of Compound 5

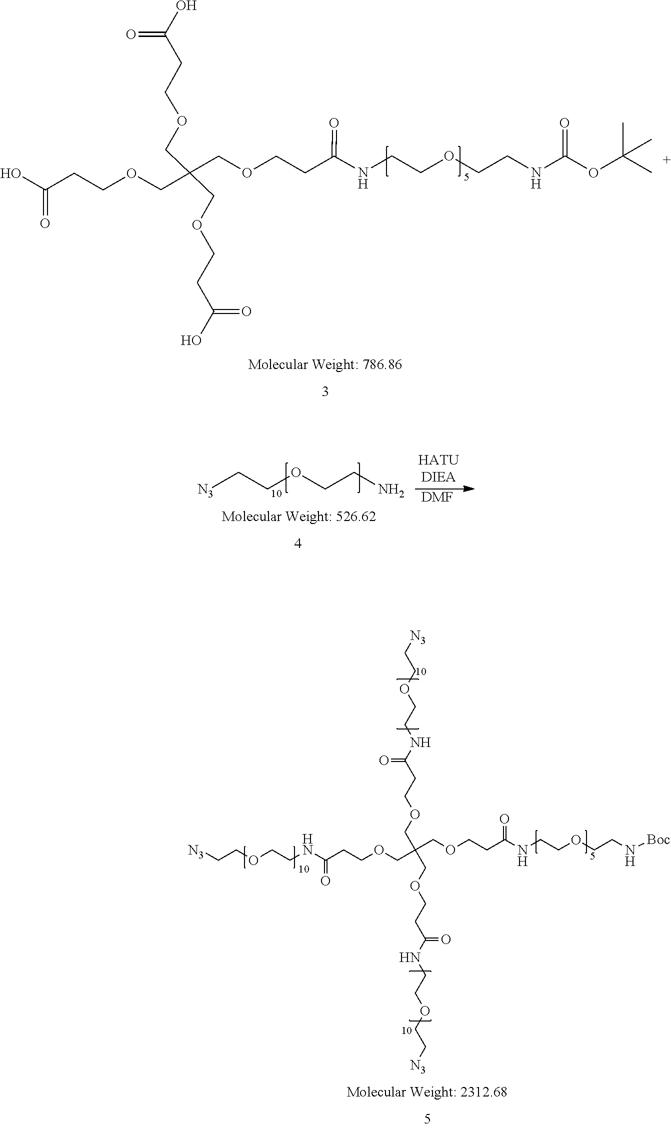

To a solution of compound 3 (20.0 mg, 25.42 μmol, 1.0 eq), compound 4 (53.5 mg, 101.67 μmol, 4.0 eq) in DMF (0.5 mL) was added HATU (28.03 mg, 73.71 μmol, 2.9 eq) and DIEA (19.7 mg, 152.51 μmol, 26.6 μL, 6.0 eq). Then the reaction mixture was stirred at 20° C. for 16 hs. LCMS showed reactant 3 was consumed completely and one main peak with desired m/z was detected (calculated MW: 2312.68, observed m/z: 1156.9 [M/2+H⁺]). The reaction mixture was diluted with 1 mL H₂O, then purified by prep-HPLC (TFA condition) to give compound 5 (40 mg, 17.30 μmol, 68.05% yield) as a colorless oil.

### Preparation of Compound 6

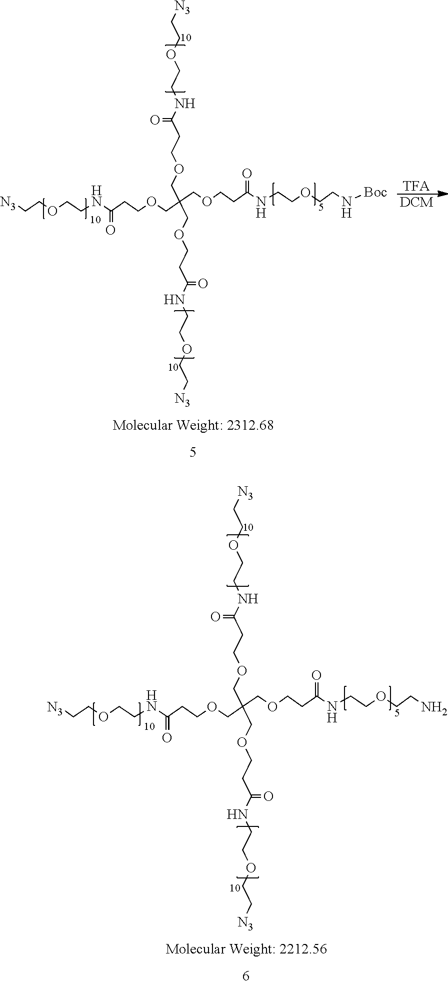

To a solution of compound 5 (40.0 mg, 17.30 μmol) in DCM (0.75 mL) was added TFA (385.0 mg, 3.38 mmol, 0.25 mL). Then the reaction mixture was stirred at 15° C. for 2 hrs. LC-MS showed one main peak with desired m/z (Calculated MW: 2212.56, observed m/z: [M/3+H⁺]738.1). The reaction mixture was diluted with 20 mL DCM then concentrated under reduced pressure five times to move TFA. The reaction mixture was used for next step directly without any purification to give compound 6 (30 mg, 13.56 μmol, 78.4% yield) as a white solid.

### Preparation of BCY9295

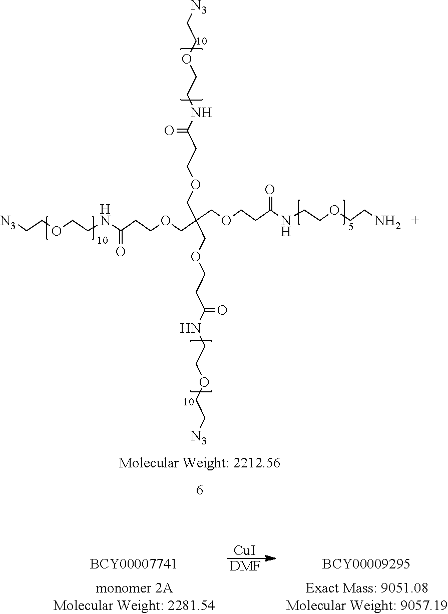

A mixture of compound 6 (30 mg, 13.56 μmol, 1.0 eq), monomer 2A (123.74 mg, 54.24 μmol, 4.0 eq), CuI (41.25 mg, 216.59 μmol, 16.0 eq) was dissolved in DMF (20 mL, solvent was degassed and purged with N₂ for 3 times), and then the mixture was stirred at 15° C. for 2 hr under N₂ atmosphere. LCMS showed desired m/z detected (calculated MW: 9057.19, observed m/z: 1510.2 [M/6+H⁺], 1294.3[M/7+H⁺], 1132.5[M/8+H⁺]), 1006.8[M/9+H⁺])). The reaction mixture was diluted with 2 mL H₂O. The residue was purified by prep-HPLC (TFA condition) to give BCY9295 (18 mg, 1.99 μmol, 53.6% purity, 14.66% yield) as a white solid.

### Preparation of BCY9932

A solution of BCY9295 in DMF (90 μL, 10 mM, 9×10⁻⁷ mol) was added to a solution of Alexa Fluor 488 in DMF (90 μL, 15 mM, 1.35×10⁻⁶ mol). DIPEA (16 μL) was added and the mixture stirred at room temperature for 1 hour. An additional 90 μL of AF488 solution in DMF (15 mM) was added and the mixture stirred overnight. The solution was diluted up to 10 mL using 6M guanidinium hydrochloride and purified using semi preparative RP-HPLC, then lyophilized to give BCY9932 as a yellow solid (1.1 mg, 1.15×10⁻⁷ mol, 12.8%) with desired m/z (Calculated MW: 9574.73, observed m/z: [M/6+H⁺] 1596.68.

### BCY7340

### Preparation of BCY7080

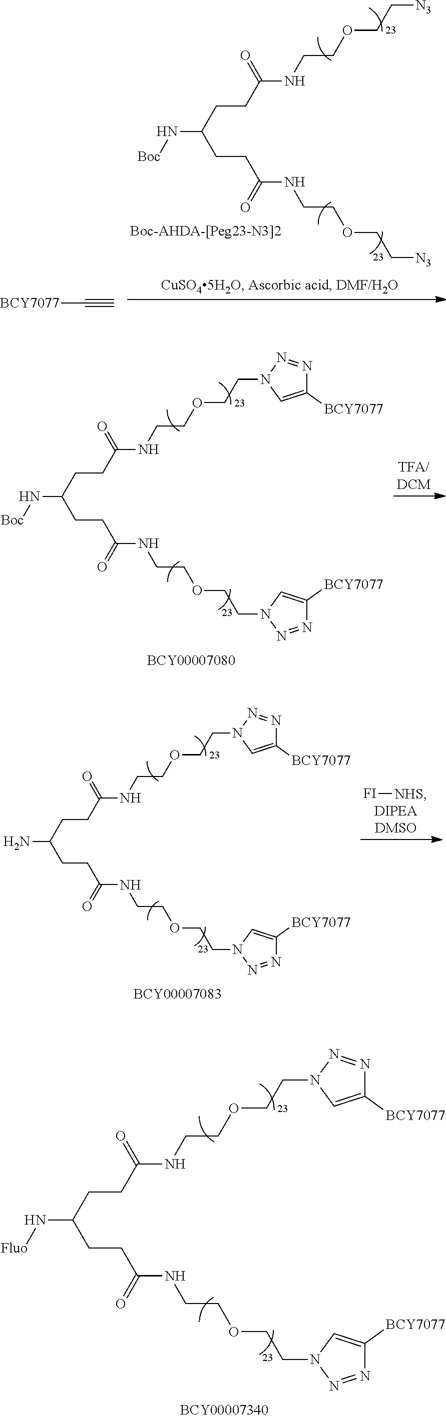

A solution of BCY7077 in DMF (1 mL, 11.1 mM, 1.1×10⁻⁵ mol) was added to a solution of Boc-AHDA-[Peg23-N3]2 (247 μL, 20 mM, 4.95×10⁻⁶ mol) and the resulting mixture purged with N₂ for 5 minutes. In a separate tube, a solution of CuSO₄.5H₂O (165 μL, 200 mM, 3.3×10⁻⁵ mol) was mixed with a solution of ascorbic acid (1.1 mL, 200 mM, 2.2×10⁻⁴ mol). Both mixtures were combined and allowed to react for 1 hour. The reaction mixture was diluted up to 9 mL with 50 mM EDTA solution and purified using preparative RP-HPLC. Fractions containing pure product were collected and lyophilised to give BCY7080 (19 mg, 2.9×10⁻⁶ mol, 59%) as a while solid.

### Preparation of BCY7083

BCY7080 (19 mg, 2.9×10⁻⁶ mol) was dissolved in 1 mL DCM/TFA and the mixture stirred for 1 hour. Solvents were blown off under a stream of N₂ and the residue dissolved in 6M guanidine hydrochloride and purified using preparative RP-HPLC. Clean fractions were pooled and lyophilised to give BCY7083 as a white solid (15.9 mg, 2.4×10⁻⁶ mol, 83%) as a white solid.

### Preparation of BCY7340

A solution of BCY7083 in DMSO (25 μL, 24.7 mM, 6.17×10⁻⁷ mol) was mixed with a solution of fluorescein-NHS (30 μL, 50 mM, 1.5×10⁻⁶ mol) and DIPEA (2.5 μL) was added. After 2 hours, the mixture was diluted to 10 mL using 6M guanidine hydrochloride and purified by preparative RP-HPLC. Samples containing clean desired material were pooled and lyophilized to give BCY7340 as a yellow solid.

BCY11467

### Preparation of Tet-[Peg10-N3]₄

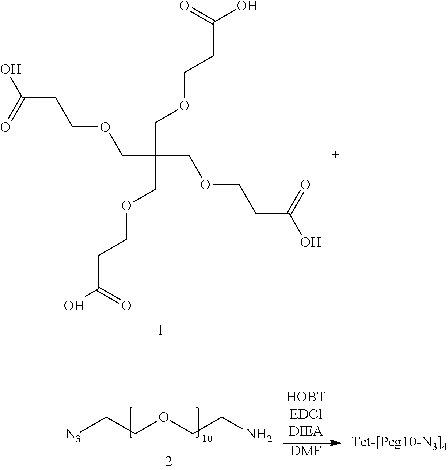

To a Solution of Compound 1 (100 mg, 235.63 μmol, 1 eq) in DMF (1 mL) was added EDCl (200 mg, 1.04 mmol, 4.43 eq) and HOBt (140 mg, 1.04 mmol, 4.4 eq) and DIPEA (185.50 mg, 1.44 mmol, 0.25 mL, 6.09 eq). Compound 2 (500 mg, 949.45 μmol, 4.03 eq) in DMF (1 mL) was added dropwise. The mixture was stirred at 25-30° C. for 12 hrs. LC-MS showed formation of a new peak as major product. The reaction mixture was purified by prep-HPLC (TFA condition) to give Tet-[Peg10-N3]₄ (385 mg, 148.75 μmol, 63.13% yield, 95% purity) as a light yellow oil.

### Preparation of Compound 3

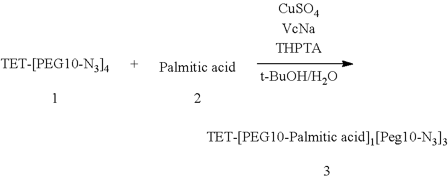

Tet-[Peg10-N3]₄ (42.0 mg, 17.1 μmol, 1.0 eq) and Compound 2 (6.0 mg, 17.1 μmol, 1.0 eq) were first dissolved in 2 mL of t-BuOH/H₂O (1:1), and then CuSO₄ (0.4 M, 45.0 μL, 1.0 eq), VcNa (9.0 mg, 45.4 μmol, 2.6 eq) and THPTA (9.0 mg, 20.7 μmol, 1.2 eq) were added. Finally 0.2 M NH₄HCO₃ was added to adjust pH to 8. All solvents here were degassed and purged with N₂ for 3 times. The reaction mixture was stirred at 40° C. for 16 hr under N₂ atmosphere. The reaction mixture was used into the next step without further purification.

### Preparation of BCY11467

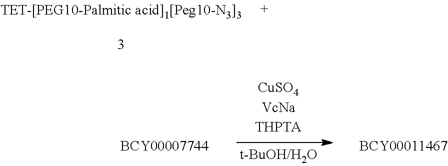

Compound 3 (32.0 mg, 11.4 μmol, 1.0 eq) and BCY7744 (90.0 mg, 38.5 μmol, 3.4 eq) were first dissolved in 2 mL of t-BuOH/H₂O (1:1), and then CuSO₄ (0.4 M, 30.0 μL, 1.0 eq), VcNa (6.0 mg, 30.3 μmol, 2.6 eq) and THPTA (10.0 mg, 23.0 μmol, 2.0 eq) were added. Finally, 0.2 M NH₄HCO₃ was added to adjust pH to 8. All solvents here were degassed and purged with N₂ for 3 times. The reaction mixture was stirred at 40° C. for 16 hr under N₂ atmosphere. LC-MS showed one main peak with desired m/z (calculated MW: 9823.29 observed m/z: 1228.7 ([M/8+H]⁺)). The reaction mixture was purified by prep-HPLC (TFA condition) and BCY11467 (4.3 mg, 0.41 μmol, 3.65% yield, 97.32% purity) was obtained as a white solid.

BCY11554

### Preparation of Compound 3

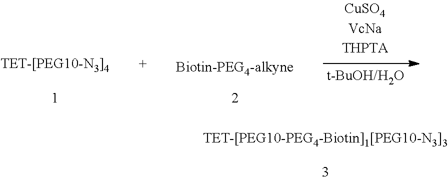

Tet-[Peg10-N3]₄ (11.0 mg, 4.5 μmol, 1.0 eq) and Compound 2 (1.0 mg, 4.4 μmol, 1.0 eq) were first dissolved in 2 mL of t-BuOH/H₂O (1:1), and then CUSO₄ (0.4 M, 12.0 μL, 1.1 eq), VcNa (2.0 mg, 10.1 μmol, 2.2 eq) and THPTA (3.0 mg, 6.9 μmol, 1.5 eq) were added. Finally, 0.2 M NH₄HCO₃ was added to adjust pH to 8. All solvents here were degassed and purged with N₂ for 3 times. The reaction mixture was stirred at 40° C. for 16 hr under N₂ atmosphere. The reaction mixture was used into the next step without further purification.

### Preparation of BCY11554

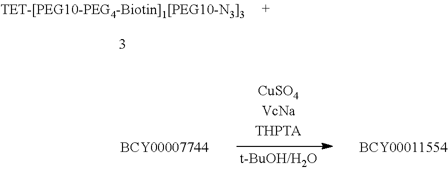

Compound 3 and BCY7744 (38.0 mg, 16.3 μmol, 3.6 eq) were first dissolved in 2 mL of t-BuOH/H₂O (1:1), and then CuSO₄ (0.4 M, 12.0 μL, 1.1 eq), VcNa (2.0 mg, 10.1 μmol, 2.2 eq) and THPTA (3.0 mg, 6.9 μmol, 1.5 eq) was added. Finally, 0.2 M NH₄HCO₃ was added to adjust pH to 8. All solvents here were degassed and purged with N₂ for 3 times. The reaction mixture was stirred at 40° C. for 16 hr under N₂ atmosphere. LC-MS showed one main peak with desired m/z (calculated MW: 9929.35, observed m/z: 1419.2 ([M/7+H]⁺) and 1242.0 ([M/8+H]⁺)). The reaction mixture was purified by prep-HPLC (TFA condition) and BCY11554 (4.3 mg, 0.43 μmol, 9.67% yield, 99.52% purity) was obtained as a white solid.

BCY11856

### Preparation of Compound 2

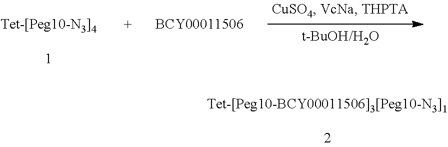

To a solution of Tet-[Peg10-N3]₄ (12 mg, 4.9 μmol, 1.0 eq), BCY11506 (35 mg, 15.0 μmol, 3.1 eq), THPTA (0.4 M, 12.2 μL, 1.0 eq) was dissolved in t-BuOH/H₂O (1:1, 2 mL, pre-degassed and purged with N₂ for 3 times), then CuSO₄ (0.4 M, 24.4 μL, 2.0 eq) and VcNa (0.4 M, 24.4 μL, 2.0 eq) were added under N₂. The pH of this solution was adjusted to 8 by dropwise addition of 0.2 M NH₄HCO₃ (in 1:1 t-BuOH/H₂O), and the solution turned to light yellow. LC-MS showed compound 1 was consumed completely and some peaks with desired m/z (MW: 9474.69, observed m/z: 1052.83 ([M/9+H]⁺), 1184.66 ([M/8+H]⁺)) was detected. The reaction mixture was concentrated under reduced pressure to remove solvent to give a residue. The residue was purified by prep-HPLC (TFA condition). Compound 2 (6.8 mg, 6.47e-1 μmol, 13.25% yield, 90.10% purity) was obtained as a white solid.

### Preparation of BCY11856

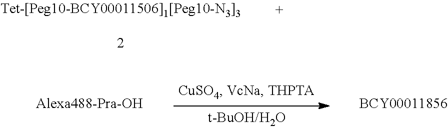

A mixture of compound 2 (6.1 mg, 6.44e-1 μmol, 1.0 eq), Alexa488-Pra-OH (0.5 mg, 6.44e-1 μmol, 1.0 eq), THPTA (0.3 mg, 6.90e-1 μmol, 1.0 eq) was dissolved in t-BuOH/H₂O (1:1, 2 mL, pre-degassed and purged with N₂ for 3 times), then CuSO₄ (0.4 M, 1.6 μL, 1.0 eq) and VcNa (0.4 M, 3.2 μL, 2.0 eq) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 40° C. for 16 hr under N₂ atmosphere. LC-MS showed Compound 2 was consumed completely and one major peak was detected on crude HPLC. The reaction mixture was directly purified by prep-HPLC (TFA condition). BCY11856 (2.5 mg, 0.24 μmol, 32% yield, 84.78% purity) was obtained as a pink solid (Calculated MW: 10044.24, observed m/z 1256.39 ([M/8+H]⁺), 1435.7 ([M/7+H]⁺)).

BCY11862

### Preparation of Tet-Peg23-N3

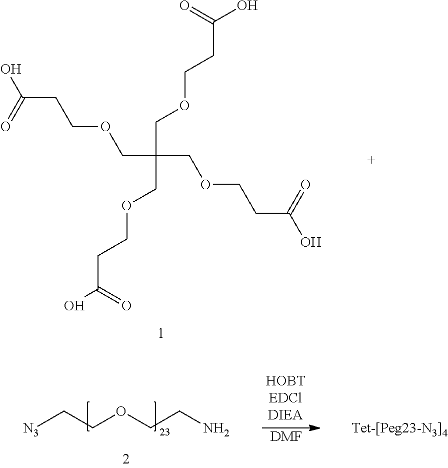

To a solution of compound 1 in DMF (1 mL) was added HOBt (56 mg, 414.45 μmol, 4.40 eq) and EDCl (80 mg, 417.32 μmol, 4.43 eq) and DIEA (73.09 mg, 565.51 μmol, 98.50 μL, 6.0 eq) then compound 2 (420 mg, 382.06 μmol, 4.05 eq) in DMF (1 mL) was added dropwise. The mixture was stirred at 20° C. for 12 hrs. LC-MS showed compound 1 was completed consumed and a new peak was formed as major product. The mixture was purified by prep-HPLC (TFA condition) to give Tet-Peg23-N3 (225 mg, 47.37 μmol, 50.26% yield, 100% purity) as a white solid.

### Preparation of Compound 3

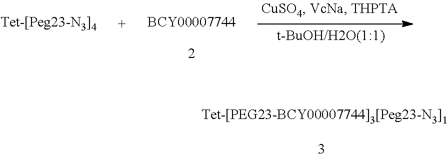

A mixture of Tet-[Peg23-N3]₄ (30 mg, 6.3 μmol, 1.0 eq.), compound 2 (44.5 mg, 19.0 μmol, 3.0 eq.), and THPTA (8.5 mg, 19.6 μmol, 3.1 eq.) was dissolved in t-BuOH/H₂O (1:1, 1 mL, pre-degassed and purged with N₂ for 3 times), and then CuSO₄ (0.4 M, 48 μL, 3.0 eq.) and VcNa (8.0 mg, 40.4 μmol, 6.4 eq.) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 40° C. for 4 hr under N₂ atmosphere. LC-MS showed Tet-[Peg23-N3]₄ was consumed completely and one main peak with desired m/z (calculated MW: 11762.46, observed m/z: 1176.96 ([(M/10+H]⁺), 1307.57 ([(M/9+H]⁺)) was detected. The reaction mixture was filtered and concentrated under reduced pressure to give a residue. The crude product was purified by prep-HPLC (TFA condition), and compound 3 (21.0 mg, 1.27 μmol, 20.18% yield, 71.4% purity) was obtained as a white solid.

### Preparation of BCY11861

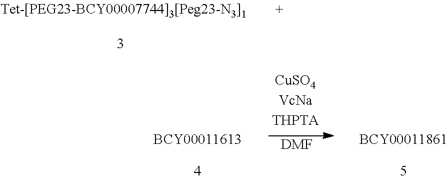

A mixture of compound 3 (21.0 mg, 1.8 μmol, 1.0 eq.), BCY11613 (4.7 mg, 2.0 μmol, 1.1 eq.), and THPTA (1.0 mg, 2.3 μmol, 1.3 eq.) was dissolved in t-BuOH/H₂O (1:1, 1 mL, pre-degassed and purged with N₂ for 3 times), and then CuSO₄ (0.4 M, 4.5 μL, 1.0 eq.) and VcNa (1.0 mg, 5.0 μmol, 2.8 eq.) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 40° C. for 2 hr under N₂ atmosphere. LC-MS showed compound 3 was consumed completely and one main peak with desired m/z (calculated MW: 14157.2, observed m/z: 1287.34 ([M/11+H]⁺), 1180.2 ([M/12+H]⁺)) was detected. The reaction mixture was filtered and concentrated under reduced pressure to give a residue. The crude product was purified by prep-HPLC (TFA condition), and BCY11861 (4.2 mg, 0.29 μmol, 16.37% yield, 98.5% purity) was obtained as a white solid.

### Preparation of BCY11862

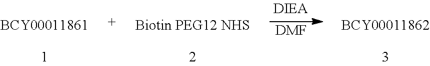

A mixture of BCY11861 (5.0 mg, 0.35 μmol, 1.0 eq.), compound 2 (0.4 mg, 0.42 μmol, 1.2 eq.), and DIEA (0.1 mg, 0.74 μmol, 0.14 μL, 2.1 eq.) was dissolved in DMF (0.5 mL). The reaction mixture was stirred at 25° C. for 1 hr. LC-MS showed compound 1 was consumed completely and one main peak with desired m/z (MW: 14983.2, observed m/z: 1500.29 ([(M/10+H⁺]), 1362.66 ([(M/11+H⁺])) was detected. The reaction mixture was concentrated under reduced pressure to remove solvent and produced a residue. The residue was then purified by prep-HPLC (neutral condition). BCY11862 (3.7 mg, 0.23 μmol, 65.79% yield, 94.1% purity) was obtained as a white solid. (MW: 14983.2, observed m/z: 1873.9180 ([(M/8+H⁺])).

BCY12239

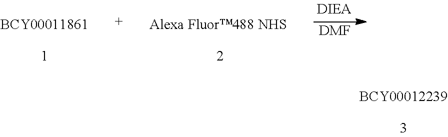

A mixture of BCY11861 (4.2 mg, 0.30 μmol, 1.0 eq.), compound 2 (0.4 mg, 0.38 μmol, 1.3 eq.) and DIEA (0.1 mg, 0.74 μmol, 0.14 μL, 2.5 eq.) was dissolved in DMF (0.5 mL). The reaction mixture was stirred at 25° C. for 1 hr. The reaction mixture was concentrated under reduced pressure to remove solvent and produced a residue. The residue was then purified by prep-HPLC (neutral condition). BCY12239 (1.8 mg, 0.12 μmol, 42.34% yield, 98.9% purity) was obtained as a pink solid. (Calculated MW: 14672.69, observed m/z: 2099.6074 ([(M/7+H⁺+H2O]).

BCY12240

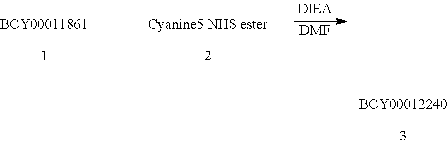

A mixture of BCY11861 (4.0 mg, 0.28 μmol, 1.0 eq.), compound 2 (0.2 mg, 0.50 μmol, 1.8 eq.), and DIEA (0.1 mg, 0.74 μmol, 0.14 μL, 2.6 eq.) was dissolved in DMF (0.5 mL). The reaction mixture was stirred at 25° C. for 1 hr. LC-MS showed compound 1 was consumed completely and one main peak with desired m/z (MW: 14622.89, observed m/z: 1219.0 ([(M/12+H⁺])) was detected. The reaction mixture was concentrated under reduced pressure to remove solvent and produced a residue. The residue was then purified by prep-HPLC (neutral condition). BCY12240 (3.4 mg, 0.21 μmol, 77.13% yield, 93.9% purity) was obtained as a light blue solid. (MW: 14622.89, observed TOF m/z: 14622.41) 1330.2719 ([(M/11+H⁺]).

BCY12380

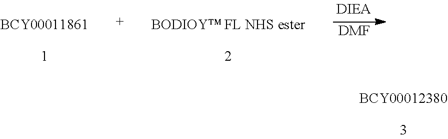

A mixture of BCY11861 (4.2 mg, 0.29 μmol, 1.0 eq.), compound 2 (0.13 mg, 0.33 μmol, 1.2 eq.), and DIEA (0.1 mg, 0.78 μmol, 0.14 μL, 2.8 eq.) was dissolved in DMF (0.5 mL). The reaction mixture was stirred at 25° C. for 1 hr. The reaction mixture was concentrated under reduced pressure to remove solvent and produced a residue. The residue was then purified by prep-HPLC (neutral condition). BCY12380 (0.8 mg, 0.21 μmol, 21.4% yield, 87.3% purity) was obtained as a light green solid. (Calculated MW: 14431.31, observed m/z: 1804.8426 ([(M/8+H⁺])

BCY 12374

### Preparation of Compound 3

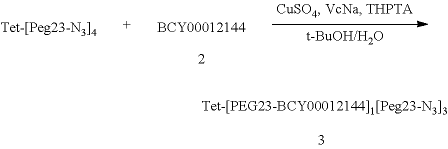

A mixture of Tet-[Peg23-Na]₄ (60 mg, 12.63 μmol, 1.0 eq.), BCY12144 (8.3 mg, 8.84 μmol, 0.7 eq.), and TH PTA (5.5 mg, 1.0 eq.) was dissolved in t-BuOH/H₂O (1:1, 1 mL, pre-degassed and purged with N₂ for 3 times), and then CuSO₄ (0.4 M, 32.0 μL, 1 0.0 eq.) and VcNa (5.5 mg, 2.0 eq.) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 40° C. for 4 hr under N₂ atmosphere. LC-MS showed BCY12144 was consumed completely and the main product peak with desired m/z (calculated MW: 7144.27, observed m/z: 1021.3 ([(M/7+H]⁺) was detected. The reaction mixture was filtered and concentrated under reduced pressure to give a residue. The crude product was purified by prep-HPLC (TFA condition), and compound 3 (21.5 mg, 2.90 μmol, 22.94% yield, 96.3% purity) was obtained as a white solid.

### Preparation of BCY12373

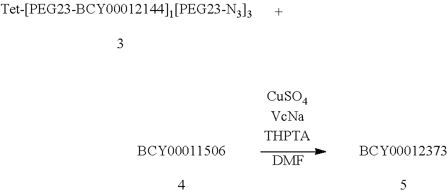

A mixture of compound 3 (21.5 mg, 3.01 μmol, 1.0 eq.), BCY11506 (21.2 mg, 9.03 μmol, 3.0 eq.), and THPTA (4.0 mg, 3.0 eq.) was dissolved in t-BuOH/H₂O (1:1, 1 mL, pre-degassed and purged with N₂ for 3 times), and then CuSO₄ (0.4 M, 23.0 μL, 3.0 eq.) and VcNa (4.0 mg, 20.2 μmol, 6.0 eq.) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 40° C. for 3 hr under N₂ atmosphere. LC-MS showed compound 3 was consumed completely and one main peak with desired m/z (calculated MW: 14160.15, observed m/z: 1288.1 ([M/11+H]⁺)), was detected. The reaction mixture was filtered and concentrated under reduced pressure to give a residue. The crude product was purified by prep-HPLC (TFA condition), and BCY12373 (28.7 mg, 1.86 μmol, 61.96% yield, 92.0% purity) was obtained as a white solid.

### Preparation of BCY12374

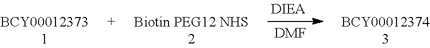

A mixture of BCY12373 (5 mg, 0.37 μmol, 1.0 eq.), compound 2 (0.4 mg, 0.37 μmol, 1.0 eq.), and DIEA (0.1 mg, 0.74 μmol, 0.14 μL, 2.0 eq.) was dissolved in DMF (0.5 mL). The reaction mixture was stirred at 25° C. for 1 hr. LC-MS showed BCY12373 was consumed completely. The reaction mixture was concentrated under reduced pressure to remove solvent and produced a residue. The residue was then purified by prep-HPLC (neutral condition). BCY12374 (2.4 mg, 0.15 μmol, 42.13% yield, 92.9% purity) was obtained as a white solid. (Calculated MW: 14983.24, observed m/z: 1873.8652 ([(M/8+H⁺])

BCY12657

### Preparation of Compound 3

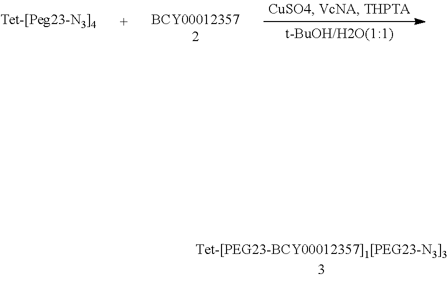

A mixture of Tet-[Peg23-N₃]₄ (150 mg, 31.6 μmol, 1.0 eq.), compound 2 (49 mg, 22.1 μmol, 0.7 eq.), and THPTA (14.0 mg, 32.2 μmol, 1.0 eq.) was dissolved in t-BuOH/H₂O (1:1, 1 mL, pre-degassed and purged with N₂ for 3 times), and then CuSO₄ (0.4 M, 80 NL, 1.0 eq.) and VcNa (13.0 mg, 65.6 μmol, 2.1 eq.) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 40° C. for 4 hr under N₂ atmosphere. LC-MS showed compound 2 was consumed completely and one main peak with was detected. The reaction mixture was filtered and concentrated under reduced pressure to give a residue. The crude product was purified by prep-HPLC (TFA condition), and compound 3 (45.8 mg, 6.17 μmol, 19.53% yield, 93.8% purity) was obtained as a white solid.

### Preparation of BCY12480

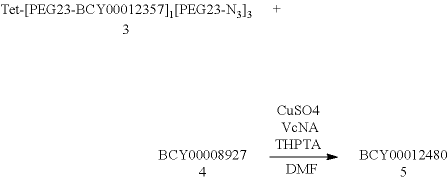

A mixture of compound 3 (45.8 mg, 6.6 μmol, 1.0 eq.), BCY8927 (42.6 mg, 19.7 μmol, 3.0 eq.), and THPTA (9.0 mg, 20.7 μmol, 3.1 eq.) was dissolved in t-BuOH/H₂O (1:1, 1 mL, pre-degassed and purged with N₂ for 3 times), and then CuSO₄ (0.4 M, 50.0 μL, 3.0 eq.) and VcNa (8.0 mg, 40.4 μmol, 6.1 eq.) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 40° C. for 3 hr under N₂ atmosphere. LC-MS showed compound 3 was consumed completely and one main peak with desired m/z (calculated MW: 13444.8, observed m/z: 1494.6 ([M/9+H]⁺), 1681.6 ([M/8+H]⁺)) was detected. The reaction mixture was filtered and concentrated under reduced pressure to give a residue. The crude product was purified by prep-HPLC (TFA condition), and BCY12480 (32.0 mg, 2.26 μmol, 34.32% yield, 96.6% purity) was obtained as a white solid.

### Preparation of BCY12657

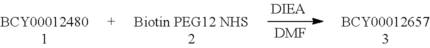

A mixture of BCY12480 (10 mg, 0.74 μmol, 1.0 eq.), compound 2 (0.7 mg, 0.74 μmol, 1.0 eq.), and DIEA (0.2 mg, 1.48 μmol, 0.3 μL, 2.0 eq.) was dissolved in DMF (0.5 mL). The reaction mixture was stirred at 25° C. for 1 hr. LC-MS showed compound 1 was consumed completely and one main peak with desired m/z (MW: 14270.8, observed m/z: 1586.3 ([(M/9+H⁺]), 1428 ([(M/10+H⁺]), 1298.6 ([(M/11+H⁺])) was detected. The reaction mixture was concentrated under reduced pressure to remove solvent and produced a residue. The residue was then purified by prep-HPLC (neutral condition). BCY12657 (4.2 mg, 0.29 μmol, 38.10% yield, 96.3% purity) was obtained as a white solid.

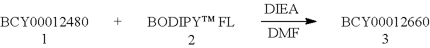

A mixture of BCY12480 (5 mg, 0.37 μmol, 1.0 eq.), compound 2 (0.2 mg, 0.51 μmol, 1.4 eq.), and DIEA (0.1 mg, 0.74 μmol, 0.14 μL, 2.0 eq.) was dissolved in DMF (0.5 mL). The reaction mixture was stirred at 25° C. for 1 hr. LC-MS showed BCY12480 was consumed completely and one main peak with desired m/z (MW: 13718.9, observed m/z: 1525.1 ([(M/9+H]⁺), 1247.6 ([(M/11+H]⁺)) was detected. The reaction mixture was concentrated under reduced pressure to remove solvent and produced a residue. The residue was then purified by prep-HPLC (neutral condition). BCY12660 (2.6 mg, 0.17 μmol, 47.69% yield, 93.6% purity) was obtained as a light green solid. (Calculated MW: 13718.9, observed m/z: 1715.8014 ([(M/8+H⁺])

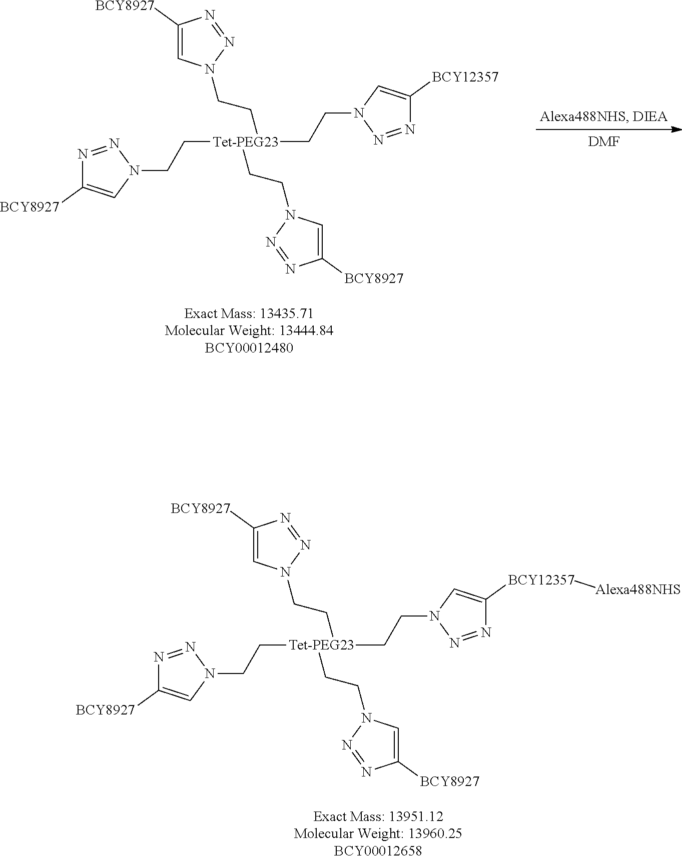

To a solution of BCY12480 (5.0 mg, 0.37 μmol, 1.0 eq) in DMF (0.5 mL) was added DIEA (0.15 mg, 1.12 μmol, 0.2 μL, 3.0 eq) and stirred for 5 min in dark. Then Alexa488-NHS (0.24 mg, 0.37 μmol, 1.0 eq) was added to the mixture under N₂ atmosphere. The mixture was stirred at 25° C. for 3 hr in dark. LC-MS showed BCY12480 was consumed completely and one main peak with desired m/z (Calculated MW: 13960.25, observed m/z: 1748.4 [(M+7H⁺+Na⁺)/8]) was detected. The reaction mixture was purified by prep-HPLC (A: 0.075% TFA in H₂O, B: ACN) to give BCY12658 (1.20 mg, 8.18e-2 μmol, 22.0% yield, 95.1% purity) as a pink solid. (Calculated MW: 13960.25, observed m/z: 1995.4625 ([(M/7+H⁺]) BCY12659

To a solution of BCY12480 (5.0 mg, 0.37 μmol, 1.0 eq) in DMF (0.5 mL) was added DIEA (0.15 mg, 1.12 μmol, 0.2 μL, 3.0 eq) and stirred for 5 min in dark. Then Cy5-NHS (0.3 mg, 0.45 μmol, 1.2 eq) was added to the mixture under N₂ atmosphere. The mixture was stirred at 25° C. for 3 hr in dark. LC-MS showed BCY12480 was consumed completely and one main peak with desired m/z (Calculated MW: 13910.49, observed m/z: 1740.1 [M/8+H]⁺) was detected. The reaction mixture was purified by prep-HPLC (A: 0.075% TFA in H₂O, B: ACN) to give BCY12659 (1.10 mg, 7.24e-2 μmol, 19.5% yield, 91.5% purity) as a light blue solid. (Calculated MW: 13910.49, observed m/z: 1265.5352 ([(M/11+H⁺])

BCY15459

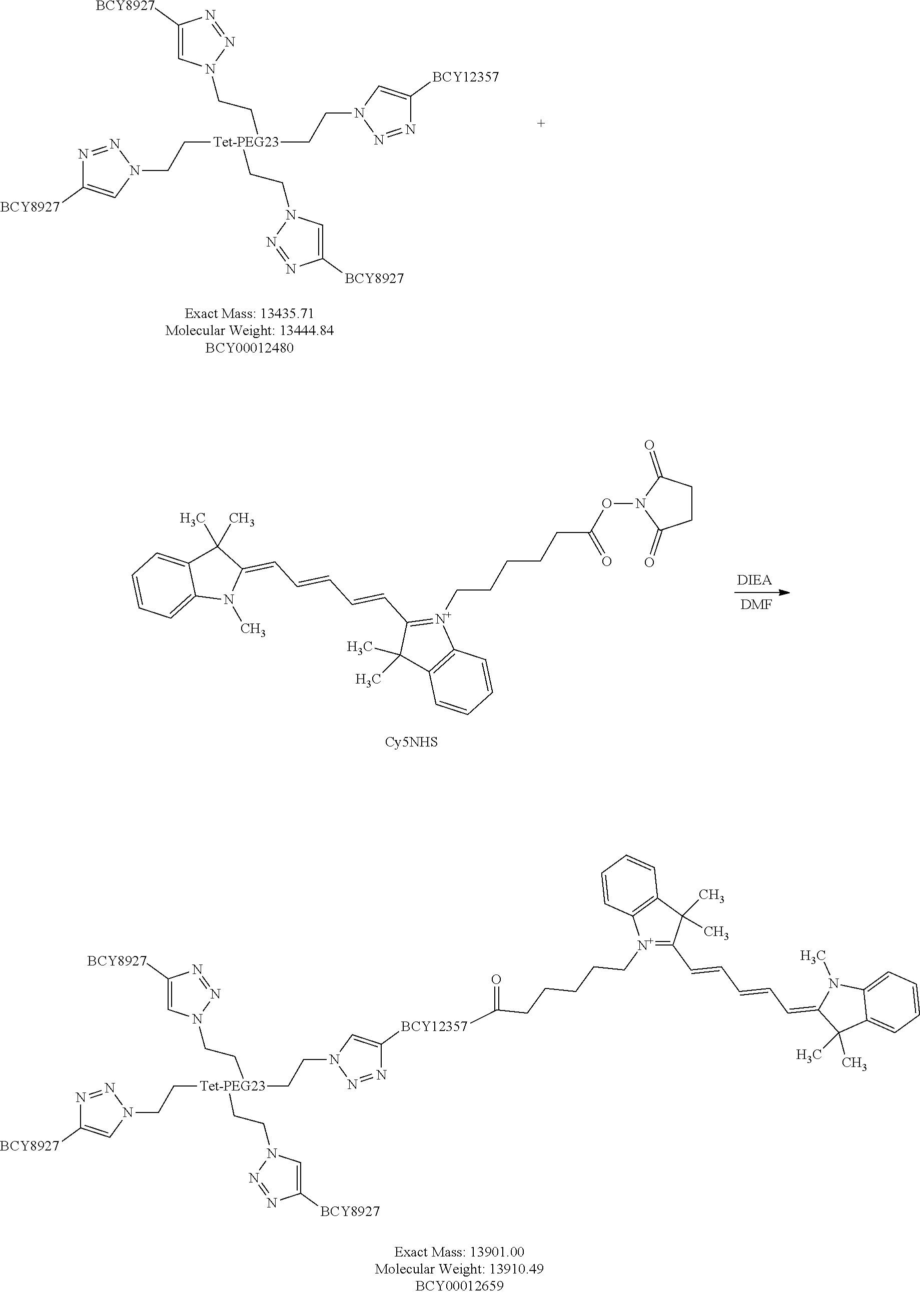

A mixture of compound 1 (10.0 mg, 18.16 μmol, 1.0 eq.), compound 2 (84.5 mg, 38.1 μmol, 2.1 eq.), and THPTA (7.9 mg, 18.16 μmol, 1.0 eq.) was dissolved in t-BuOH/0.2 M (aq) NH₄HCO₃ (1:1, 0.5 mL), degassed and purged with N₂, and then aqueous solution of CuSO₄ (0.4 M, 45.4 μL, 1.0 eq.) and Vc (14.5 mg, 72.64 μmol, 4.0 eq.) were added under N₂. The pH of this solution was adjusted to 8, and the solution turned light yellow. The reaction mixture was stirred at 25 00 for 2 hr under N₂ atmosphere. LC-MS showed BP-24369 was consumed completely and one main peak with desired m/z was detected. The reaction mixture was filtered and concentrated under reduced pressure to give a residue. The crude product was purified by preparative HPLC, and BCY15459 (55 mg, 10.48 umol, 57.71% yield, 97.5% purity) was obtained as a white solid. Calculated MW: 4985.8, observed m/z: 1247.7 [M+4H]⁴⁺, 998.4 [M+5H]⁵⁺.

BCY15416

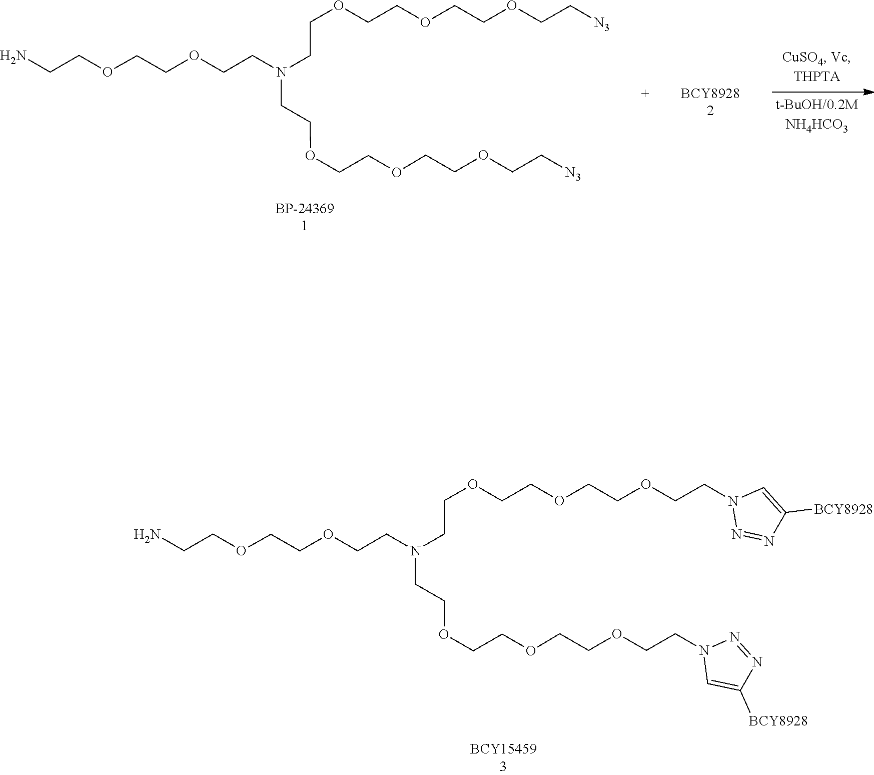

A mixture of compound 1 (20.0 mg, 4.01 μmol, 1.0 eq.), compound 2 (5.0 mg, 8.02 μmol, 2.0 eq.) was dissolved in DMF (0.5 mL). The pH of this solution was adjusted to 8 by dropwise addition of DIEA (5.2 mg, 40.11 μmol, 7.0 μL, 10 eq.). The reaction mixture was stirred at 25° C. for 1 hr. LC-MS showed BCY15459 remained (calculated MW: 4985.8, observed m/z: 1247.9 ([M/4+H]⁺), 998.1 ([M/5+H]⁺). Then additional compound 2 (5.0 mg, 8.02 μmol, 2.0 eq.) was added to the reaction mixture, and the reaction mixture was stirred at 25° C. for 1 hr. LC-MS showed BCY15459 was consumed completely, and one main peak with desired m/z (calculated (MW: 5502.27, observed m/z: 1376.8 ([M/4+H]⁺), 1100.8 ([M/5+H]⁺)) was detected. The reaction mixture was filtered and concentrated under reduced pressure to give a residue. The crude product was purified by preparative HPLC, and BCY15416 (9.8 mg, 1.72 μmol, 42.76% yield, 96.3% purity) was obtained as a red solid.

BCY15985

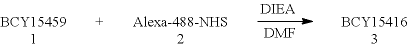

A mixture of compound 1 (30.0 mg, 6.02 μmol, 1.0 eq.), compound 2 (8.49 mg, 9.03 μmol, 1.5 eq.) was dissolved in DMF (0.5 mL). The pH of this solution was adjusted to 8 by dropwise addition of 0.1 M DIEA (7.7 mg, 60.17 μmol, 10.5 μL, 10.0 eq.). The reaction mixture was stirred at 25 HC for 1.0 hr. LC-MS showed BCY15459 was consumed completely, and one main peak with desired m/z (calculated MW: 5811.8, observed m/z: 1454.3 ([M/4+H]⁺), 1163.4 ([M/5+H]⁺), 969.7 ([M/6+H]⁺), 831.4 ([M/7+H]⁺)) was detected, and BCY15459-2 eq. Biotion-PEG12-NHS (calculated MW: 6637.8, observed m/z: 1328.7 ([M/5+H]⁺)) was also observed. The reaction mixture was filtered and concentrated under reduced pressure to give a residue. The crude product was purified by prep-HPLC (TFA condition), and BCY15985 (21 mg, 3.53 μmol, 58.61% yield, 97.6% purity) was obtained as a white solid.

### BCY13390

Procedure for Preparation of BCY12476

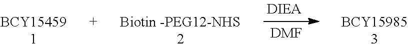

A mixture of N-(acid-PEG3)-N-bis(PEG3-azide) (70.0 mg, 112.2 μmol, 1.0 eq), HATU (51.2 mg, 134.7 μmol, 1.2 eq) and DIEA (29.0 mg, 224.4 μmol, 40 μL, 2.0 eq) was dissolved in DMF (2 mL), and mixed for 5 min. Then BCY8116 (294.0 mg, 135.3 μmol, 1.2 eq) was added. The reaction mixture was stirred at 40° C. for 16 hr. LC-MS showed one main peak with desired m/z. The reaction mixture was concentrated under reduced pressure to remove solvent and produced a residue. The residue was then purified by preparative HPLC. BCY12476 (194.5 mg, 66.02 μmol, 29% yield, 94% purity) was obtained as a white solid. Calculated MW: 2778.17, observed m/z: 1389.3 ([M+2H]²⁺), 926.7 ([M+3H]³⁺).

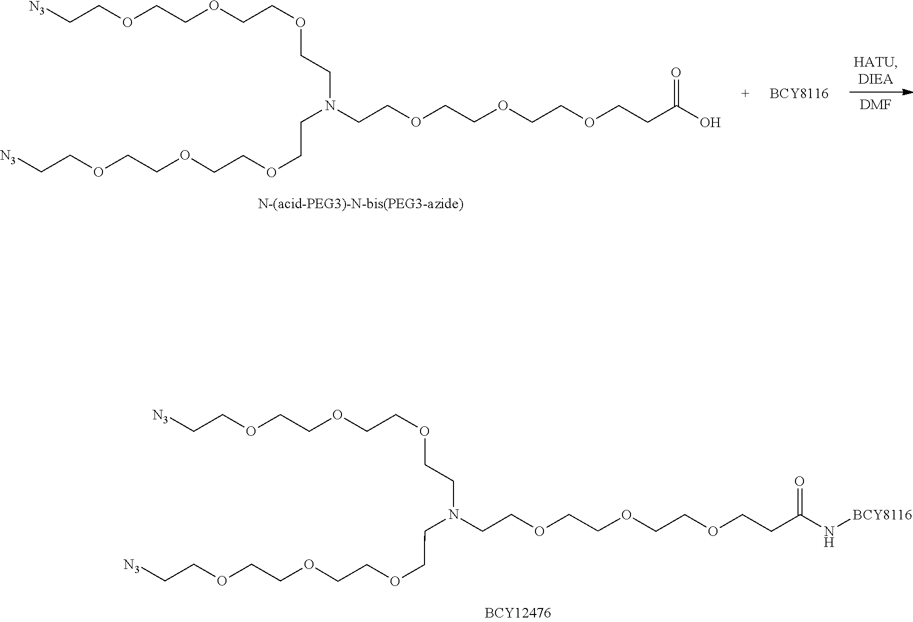

### Procedure for Preparation of BCY13689

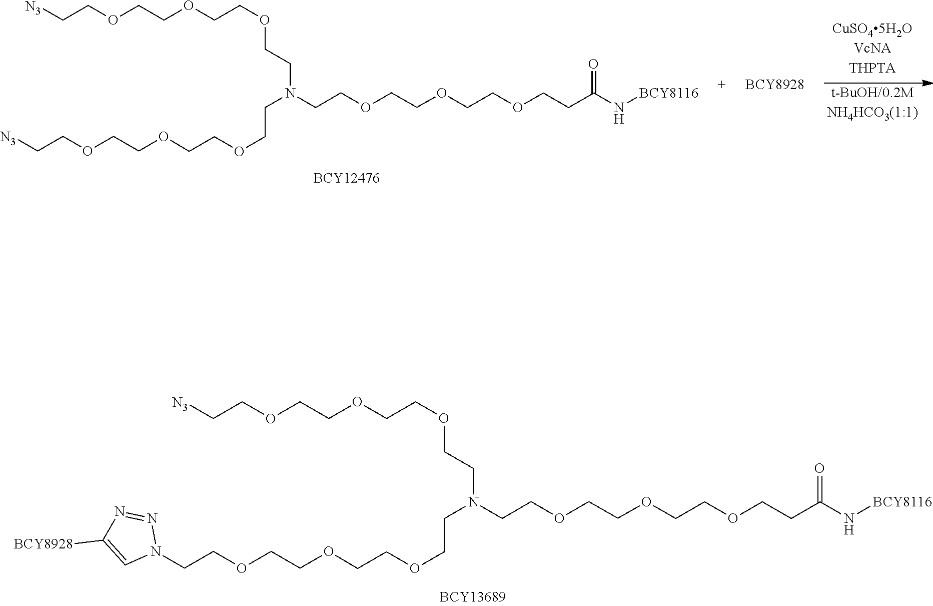

A mixture of BCY12476 (47.0 mg, 16.91 μmol, 1.0 eq), BCY8928 (30.0 mg, 13.53 μmol, 0.8 eq), and THPTA (36.7 mg, 84.55 μmol, 5.0 eq) was dissolved in t-BuOH/H₂O (1:1, 8 mL, pre-degassed and purged with N₂), and then CuSO₄ (0.4 M, 21.0 μL, 0.5 eq) and VcNa (67.0 mg, 338.21 μmol, 20.0 eq) were added under N₂. The pH of this solution was adjusted to 8 by dropwise addition of 0.2 M NH₄HCO₃ (in 1:1 t-BuOH/H₂O), and the solution turned light yellow. The reaction mixture was stirred at 25° C. for 1.5 h under N₂ atmosphere. LC-MS showed that some BCY12476 remained, BCY8928 was consumed completely, and a peak with the desired m/z was detected. The reaction mixture was filtered and concentrated under reduced pressure to give a residue. The crude product was purified by preparative HPLC, and BCY13689 (25.3 mg, 4.56 μmol, 27% yield, 90% purity) was obtained as a white solid. Calculated MW: 4995.74, observed m/z: 1249.4 ([M+4H]⁴⁺), 999.9 ([M+5H]⁵⁺).

### Procedure for Preparation of BCY13390

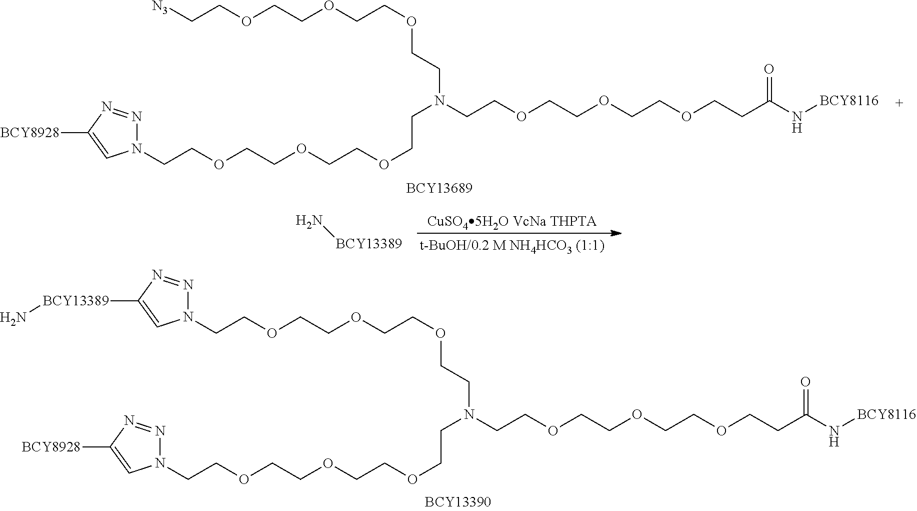

A mixture of BCY13689 (43.6 mg, 8.73 μmol, 1.0 eq), BCY13389 (20.8 mg, 9.16 μmol, 1.05 eq), and THPTA (3.8 mg, 8.73 μmol, 1.0 eq) was dissolved in t-BuOH/H₂O (1:1, 1 mL, pre-degassed and purged with N₂), and then CuSO₄ (0.4 M, 22.0 μL, 1.0 eq) and VcNa (3.5 mg, 17.45 μmol, 2.0 eq) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25° C. for 2 hr under N₂ atmosphere. LC-MS showed a significant peak corresponding to the desired m/z. The reaction mixture was filtered and concentrated under reduced pressure to give a residue. The crude product was purified by preparative HPLC, and BCY13390 (33.8 mg, 4.21 μmol, 48% yield, 90% purity) was obtained as a white solid. Calculated MW: 7270.41, observed m/z: 1454.9 ([M+5H]⁵⁺), 1213.2 ([M+6H]⁶⁺)

BCY13582

### Procedure for Preparation of BCY13582

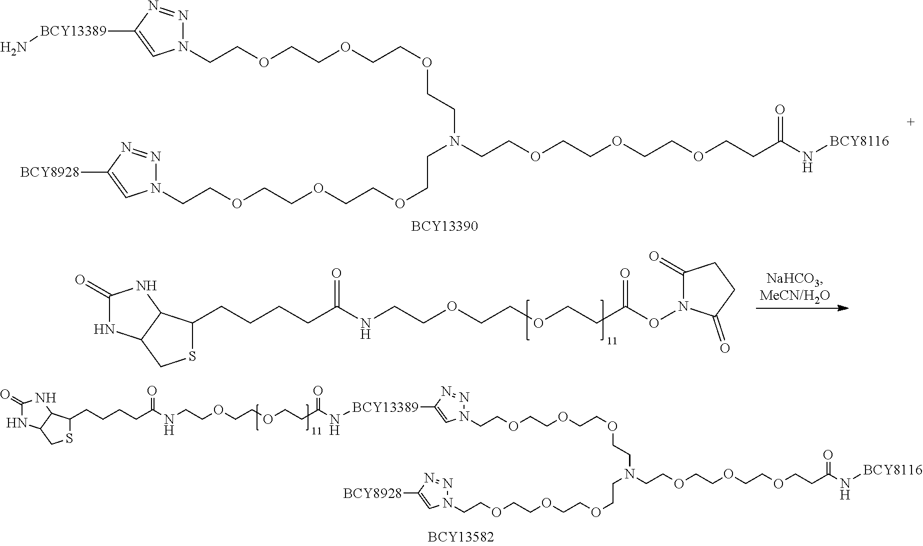

A mixture of BCY13390 (5.0 mg, 0.6 μmol, 1.0 eq), biotin-PEG12-NHS ester (CAS 365441-71-0, 0.7 mg, 0.72 μmol, 1.1 eq) was dissolved in MeCN/H₂O (1:1, 2 mL). The pH of this solution was adjusted to 8 by dropwise addition of 1.0 M NaHCO₃. The reaction mixture was stirred at 25° C. for 0.5 hr. LC-MS showed BCY13390 was consumed completely, and one main peak with desired m/z was detected. The reaction mixture was filtered and concentrated under reduced pressure to give a residue. The crude product was purified by preparative HPLC, and BCY13582 (2.5 mg, 0.30 μmol, 43% yield, 96% purity) was obtained as a white solid. Calculated MW: 8096.43, observed m/z: 1351.1 ([M+6H]⁶⁺), 1158.5 ([M+7H]⁷⁺).

BCY 13583

### Procedure for Preparation of BCY13583

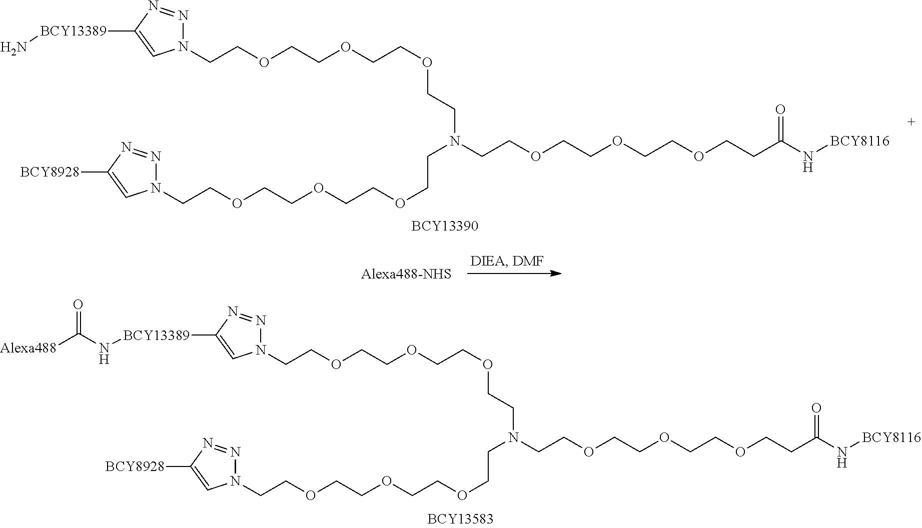

A mixture of BCY13390 (15.0 mg, 2.06 μmol, 1.0 eq) and Alexa Fluor® 488 NHS ester (2.5 mg, 4.12 μmol, 2.0 eq) was dissolved in DMF (0.5 mL). DIEA (2.6 mg, 20.63 μmol, 3.6 μL, 10 eq) was then added dropwise. The reaction mixture was stirred at 25° C. for 1 hr. LC-MS showed BCY13390 remained, and one main peak with desired m/z was detected. Additional Alexa Fluor® 488 NHS ester (2.0 mg, 3.09 μmol, 1.5 eq) was added to the reaction mixture, and the reaction mixture was stirred at 25° C. for one additional hour. HPLC showed BCY13390 was consumed completely. The reaction mixture was filtered and concentrated under reduced pressure to give a residue. The crude product was purified by preparative HPLC, and BCY13583 (5 mg. 0.61 μmol. 29% yield, 95% purity) was obtained as a red solid. Calculated MW: 7787.9, observed m/z: 1948.8 ([M+4H+H₂O]⁴⁺), 1558.6 ([M+5H+H₂O]⁵⁺) 1299.1 ([M+7H+H₂O]⁷⁺).

### Procedure or Preparation of BCY13628

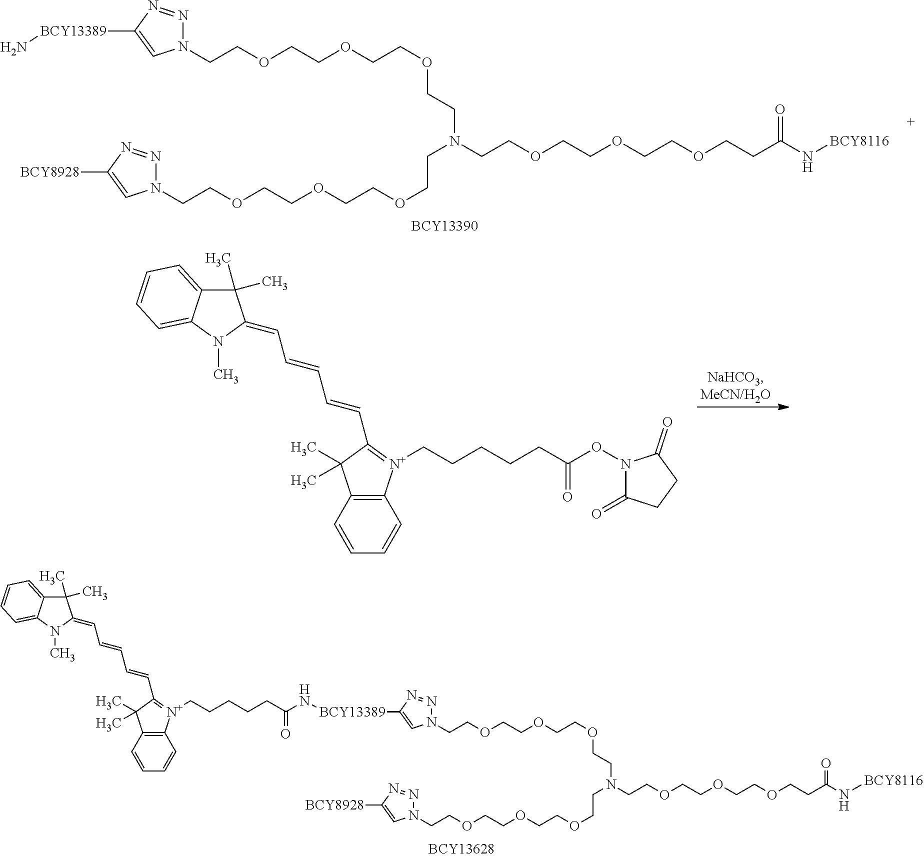

A mixture of BCY13390 (5.6 mg, 0.77 μmol, 1.0 eq) and cyanine 5 NHS ester (0.5 mg, 0.85 μmol, 1.1 eq) was dissolved in MeCN/H₂O (1:1, 2 mL). The pH of this solution was adjusted to 8 by dropwise addition of 1.0 M NaHCO₃. The reaction mixture was stirred at 25° C. for 0.5 hr. LC-MS showed BCY13390 was consumed completely and one main peak with desired m/z was detected. The reaction mixture was filtered and concentrated under reduced pressure to give a residue. The crude product was purified by preparative HPLC, and BCY13628 (2.9 mg, 0.36 μmol, 46% yield, 95% purity) was obtained as a blue solid. Calculated MW: 7736.06, observed m/z: 1289.9 ([M+6H]⁶⁺), 1105.5 ([M+7H]⁷⁺).

Procedure for preparation of BCY14414

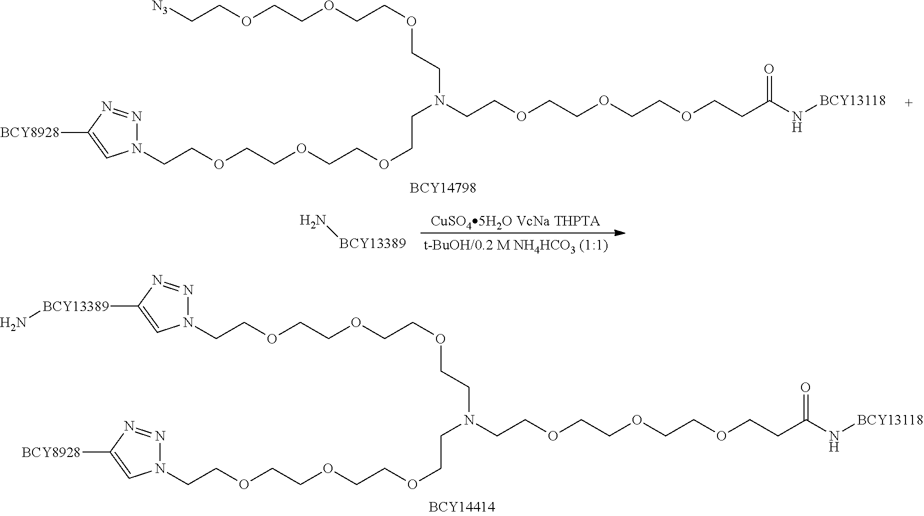

A mixture of BCY14798 (21.0 mg, 4.02 μmol, 1.0 eq), BCY13389 (10.0 mg, 4.42 μmol, 1.1 eq), and THPTA (1.8 mg, 4.02 μmol, 1.0 eq) was dissolved in t-BuOH/0.2 M NH₄HCO₃ (1:1, 0.5 mL, pre-degassed and purged with N₂), and then CuSO₄ (0.4 M, 5.0 μL, 0.5 eq) and sodium ascorbate (2.8 mg, 16.06 μmol, 4.0 eq) were added under N₂. The pH of this solution was adjusted to 7.5 by dropwise addition of 0.2 M NH₄HCO₃ (in 1:1 t-BuOH/0.2 M NH₄HCO₃) and the solution turned to light yellow. The reaction mixture was stirred at 25° C. for 2 hr under N₂ atmosphere. LC-MS showed BCY14798 was consumed completely, some BCY13389 remained and one main peak with desired m/z was detected. The reaction mixture was filtered and concentrated under reduced pressure to give a residue. The crude product was purified by preparative and BCY14414 (20 mg, 2.40 μmol, 59.73% yield, 90.9% purity) was obtained as a white solid. Calculated MW: 7503.74, observed m/z: 1251.5 ([M+5H]⁵⁺), 1072.9 ([M+7H]⁷⁺).

BCY14417

### Procedure for Preparation of BCY14417

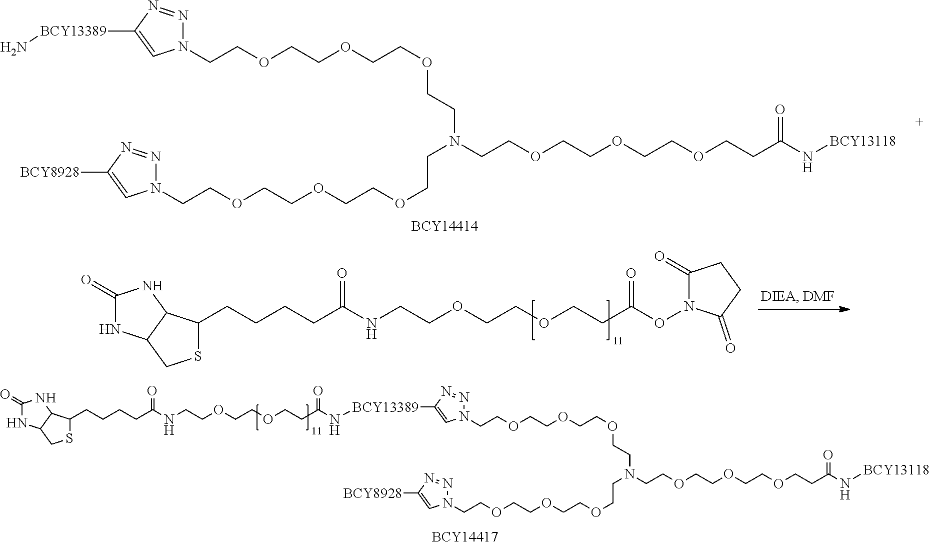

A mixture of BCY14414 (13.0 mg, 1.73 μmol, 1.0 eq) and biotin-PEG12-NHS ester (CAS 365441-71-0, 4.2 mg, 4.50 μmol, 2.6 eq) was dissolved in DMF (0.5 mL). The pH of this solution was adjusted to 8 by dropwise addition of DIEA. The reaction mixture was stirred at 25° C. for 0.5 hr. LC-MS showed BCY14414 was consumed completely, and one main peak with desired m/z was detected. The reaction mixture was filtered and concentrated under reduced pressure to give a residue. The crude product was purified by preparative HPLC and BCY14417 (9.0 mg, 1.07 μmol, 80.49% yield, 90.8% purity) was obtained as a white solid. Calculated MW: 8329.74, observed m/z: 1389.6 ([M+6H]⁶⁺), 1191.9 ([M+7H]⁷⁺).

### Procedure for Preparation of BCY14418

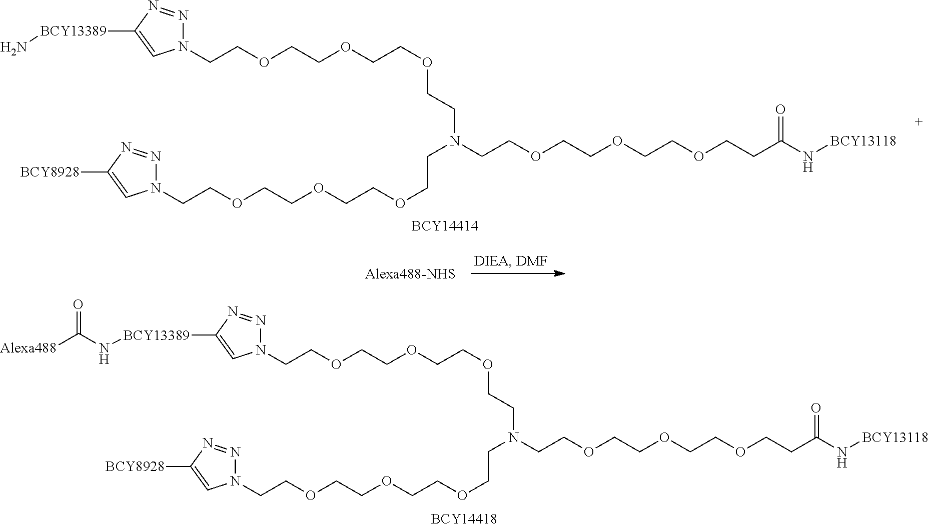

A mixture of BCY14414 (5.6 mg, 0.75 μmol, 1.0 eq) and Alexa Fluor® 488 (0.9 mg, 1.49 μmol, 2.0 eq) was dissolved in DMF (0.3 mL). Then pH of this solution was adjusted to 8 by dropwise addition of DIEA. The reaction mixture was stirred at 25 00 for 1.0 hr. LC-MS showed BCY14414 was consumed completely, and one main peak with desired m/z was detected. The reaction mixture was filtered and concentrated under reduced pressure to give a residue. The crude product was purified by preparative HPLC, and BCY14418 (2.3 mg, 0.25 μmol, 32.89% yield, 85.6% purity) was obtained as a red solid. Calculated MW: 8020.19, observed m/z: 1337.2 ([M+6H]⁶⁺).

### Procedure for Preparation of BCY9594-BP-23825-BCY8928

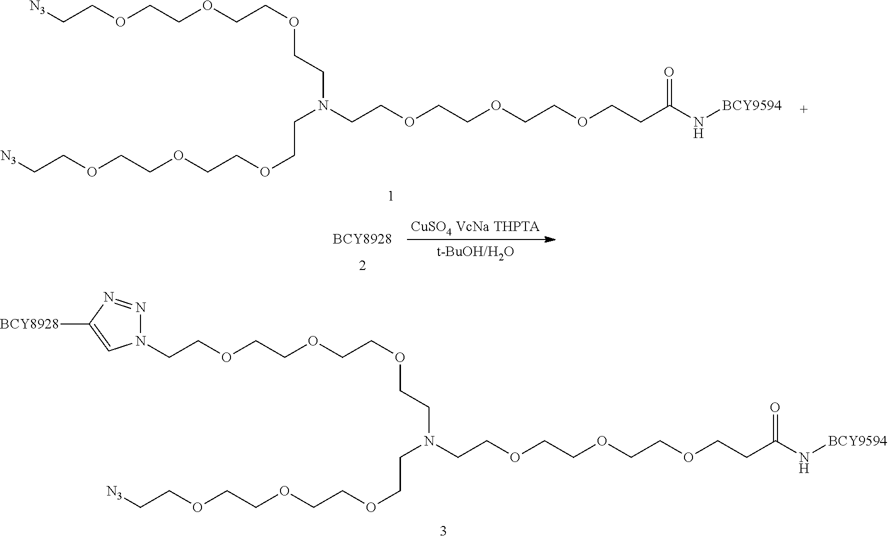

A mixture of compound 1 (50.0 mg, 16.6 μmol, 1.0 eq.), compound 2 (29.5 mg, 13.3 μmol, 0.8 eq.), and THPTA (36.1 mg, 83.1 μmol, 5.0 eq.) was dissolved in t-BuOH/H₂O (1:1, 8 mL, degassed and purged with N₂), and then aqueous solution of CuSO₄ (0.4 M, 20.8 μL, 0.5 eq.) and VcNa (65.9 mg, 332.6 μmol, 20.0 eq.) were added under N₂. The pH of this solution was adjusted to 7.5 by dropwise addition of 0.5 mL 0.2 M NH₄HCO₃ (in 1:1 t-BuOH/H₂O), and the solution turned light yellow. Then the reaction mixture was stirred at 25° C. for 24 hr under N₂ atmosphere. The reaction was set up for two batches in parallel. LC-MS showed compound 1 and little amount of compound 2 remained, and desired m/z was detected. The reaction mixture was filtered to remove the insoluble residue. The crude product was purified by preparative HPLC, and compound 3 (31.5 mg, 5.44 μmol, 16.36% yield, 90.22% purity) was obtained as a white solid. Calculated MW: 5224.07, observed m/z: 1306.9 ([M+4H]⁴⁺), 871.6 ([M+6H]⁶⁺)

### Procedure for Preparation of BCY14413

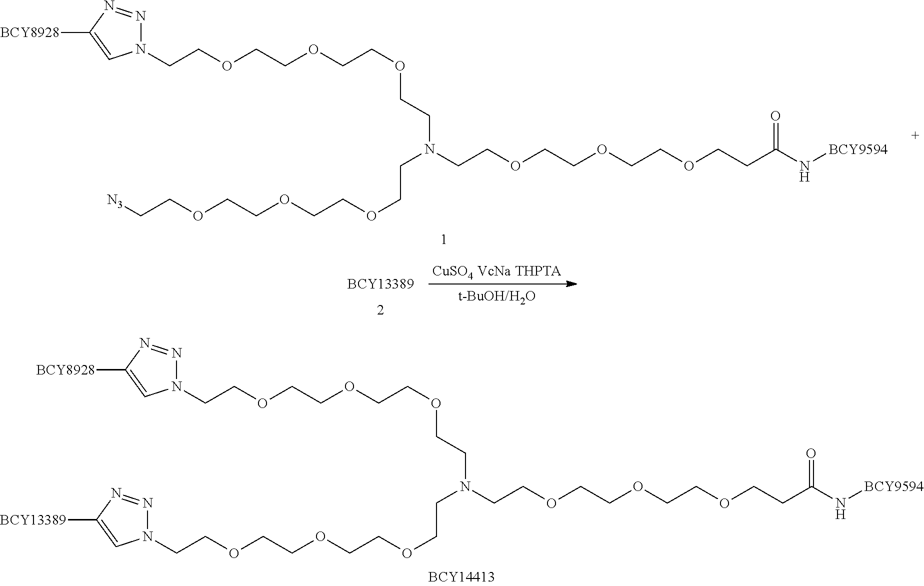

A mixture of compound 1 (31.5 mg, 6.03 μmol, 1.0 eq.), compound 2 (14.4 mg, 6.33 μmol, 1.05 eq.), and THPTA (2.62 mg, 6.03 μmol, 1.0 eq.) was dissolved in t-BuOH/H₂O (1:1, 1.0 mL, degassed and purged with N₂), and then aqueous solution of CuSO₄ (0.4 M, 15.07 μL, 1.0 eq.) and VcNa (4.78 mg, 24.12 μmol, 4.0 eq.) were added under N₂. The pH of this solution was adjusted to 7.5 by dropwise addition of 0.5 mL 0.2 M NH₄HCO₃ (in 1:1 t-BuOH/H₂O), and the solution turned light yellow. Then the reaction mixture was stirred at 25° C. for 3 hrs under N₂ atmosphere. LC-MS showed little amount of compound 2 remained, compound 1 was consumed completely, and one main peak with desired m/z was detected. The reaction mixture was filtered to remove the insoluble residue. The crude product was purified by preparative HPLC, and BCY14413 (22.5 mg, 3.00 μmol, 43.10% yield, 86.63% purity) was obtained as a white solid. Calculated MW: 7498.75, observed m/z: 938.3 ([M+8H]⁸⁺), 1072.2 ([M+7H]⁷⁺), 1250.9 ([M+6H]⁶⁺), 1500.8 ([M+5H]⁵⁺).

BCY14415

### Procedure for Preparation of BCY14415

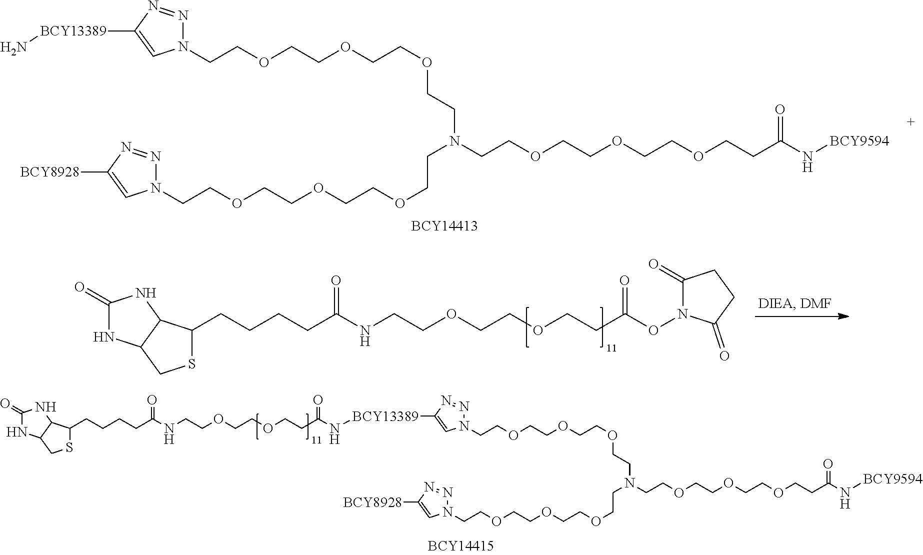

A mixture of BCY14413 (10.0 mg, 1.33 μmol, 1.0 eq.) and biotin-Peg12-NHS (2.6 mg, 2.80 μmol, 2.6 eq.) was dissolved in DMF (0.3 mL). The pH of this solution was adjusted to 8 by dropwise addition of DI EA. The reaction mixture was stirred at 25° C. for 0.5 hr. LC-MS showed BCY14413 was consumed completely, and one main peak with desired m/z was detected. The reaction mixture was filtered and concentrated under reduced pressure to give a residue. The crude product was purified by preparative HPLC, and BCY14415 (10 mg, 1.07 μmol, 80.49% yield, 90.2% purity) was obtained as a white solid. Calculated MW: 8324.73, observed m/z: 1388.4 ([M+6H]⁶⁺), 1190.2 ([M+7H]⁷⁺), 1041.5 ([M+8H]⁸⁺), 926.0 ([M+9H]⁹⁺)

### Procedure for Preparation of BCY14416

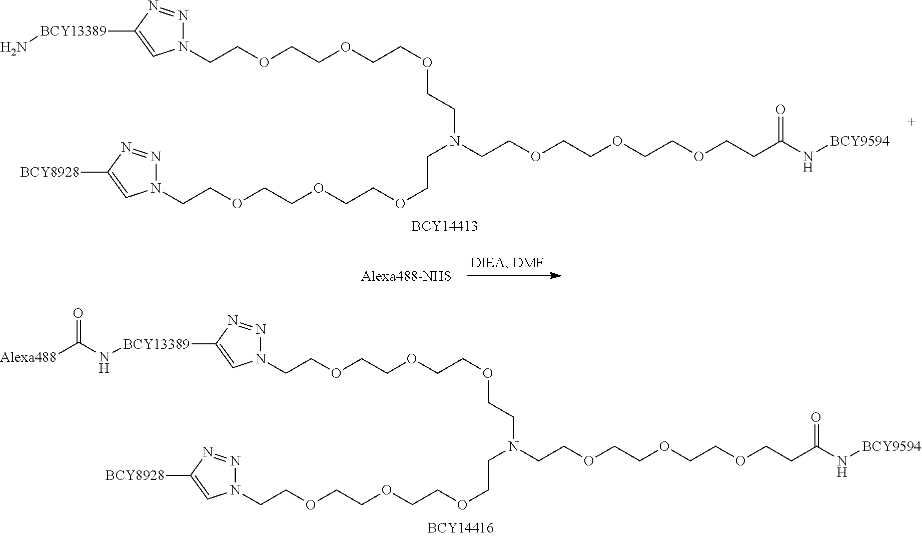

A mixture of compound BCY14413 (5.1 mg, 0.68 μmol, 1.0 eq.) and Alexa Fluor 488 NHS ester (0.5 mg, 8.16e-1 μmol, 1.2 eq.) was dissolved in DMF (0.3 mL). The pH of this solution was adjusted to 8 by dropwise addition of DIEA. The reaction mixture was stirred at 25° C. for 0.5 hr. LC-MS showed that some BCY14413 remained and one main peak with desired m/z was detected. The reaction mixture was filtered and concentrated under reduced pressure to give a residue. The crude product was purified by preparative HPLC, and the main peak was collected as two fractions with different purity, and BCY14416 (0.7 mg, 0.065 μmol, 9.84% yield, 96.4% purity) and (0.5 mg, 0.047 μmol, 7.03% yield, 91.2% purity) were obtained as red solid. Calculated MW: 8015, observed m/z: 1336.5 ([M+7H]⁷⁺).

### Preparation of Non-Modified Multimeric Binding Complexes

The following non-modified multimeric binding complexes were prepared exactly as described in PCT/GB2019/050485:

TABLE 4
Non-Modified Multimeric Binding Complexes

| Multimer | Corre- | Number | Central | | |
|---|---|---|---|---|---|
| Compound | sponding | of | Hinge | Spacer | Attachment |
| Number | Monomer | Monomers | Moiety | Molecule | Point |
| BCY7839 | BCY7744 | 3 | B | S_{A}: | D-Lys(PYA)₄ |
| | | | | n = 10 | |
| BCY7842 | BCY7744 | 4 | A | S_{A}: | D-Lys(PYA)₄ |
| | | | | n = 23 | |
| BCY8945 | BCY8927 | 4 | A | S_{A}: | Lys(PYA)₃ |
| | | | | n = 23 | |
| BCY11451 | BCY11506 | 4 | A | S_{A}: | L-Lys(PYA)₄ |
| | | | | n = 23 | |

BCY12491 was prepared as described in Example 2 of PCT/GB2020/051831.

### Biological Data

### 1. CD137 Reporter Cell Activity Washout Assay

Jurkat cells engineered to overexpress CD137 and express a luciferase gene under the NF-κB promoter were purchased from Promega. The reporter cells were incubated with 10 nM of CD137 agonists for the indicated times at 37° C. in RPMI1640 media with 1% FBS. After either 30, 60, or 120 minutes, cells were washed in an excess of culture media and resuspended in 75 μL of fresh media or no wash was performed (no washout). All washout conditions were performed in duplicate. Cells then continued to incubate for a total of 6 hours (an additional 5.5, 5, or 4 hours respective to exposure times). After incubation, 75 μL of Bio-Glo reagent (Promega) was added to each well and allowed to equilibrate for 10 minutes at room temperature. Luminescence was read on the Clariostar plate reader (BMG LabTech). Fold induction was calculated by dividing the luminescence signal by background wells (reporter cells with no agonist added). Data was graphed in Prism and is displayed as a bar graph of the means or replicates with standard deviation error bars.

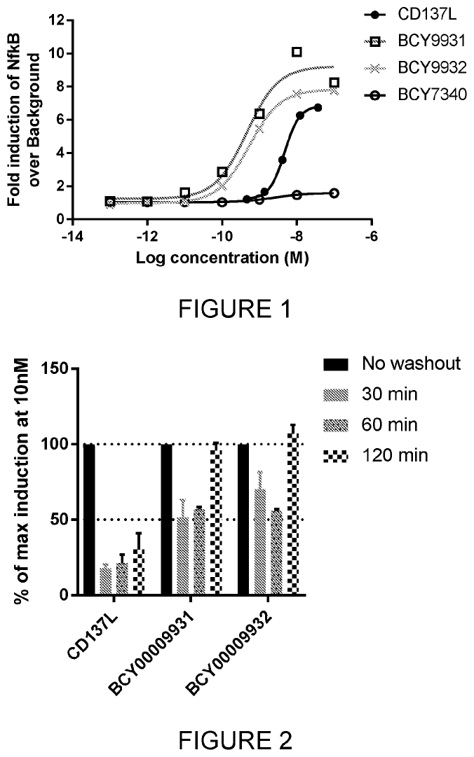

The data presented in FIG. 2 demonstrates that fluorescent CD137 multimers (BCY9931 and BCY9932) maintain CD137 agonism activity after washout consistent with highly avid binding to the trimeric CD137 receptor complex. The data in FIG. 1 shows that the fluorescent CD137 multimers BCY9931 and BCY9932 display dose responsive induction of NFkB-luciferase activity in the CD137 reporter cell assay. They are shown in comparison to the activity of CD137 ligand.

### 2. Fluorescent Multimers Human Immune Cell Direct Binding Assay

Human peripheral blood mononuclear cells (PBMCs) were isolated from buffy coats from two donors according to standard protocols. PBMCs were used for the next step or alternatively CD8+ T cells were then isolated from the PBMCs with a MACS CD8+ T cell isolation kit (negative bead selection) according to the manufacturer's protocol (Miltenyi Biotec). PBMCs or purified CD8+ T cells were then activated for 24 hours in tissue culture flasks coated with CD3 antibody in R10 media (RPMI1640+10% FBS+1× Pen-Strep). Alternatively, cells were incubated in R10 media containing 1 μg/ml of soluble CD3 antibody (OKT3) for 24 hours. Cells were then transferred to CD3-coated 96-well flat bottom plates and incubated in R10 media in the presence of fluorescent CD137 multimers or a fluorescent EphA2 bicyclic peptide monomer (BCY0215), which was used as a negative control, at different concentrations for 1 hour at 37° C. After 1 hour, cells were transferred to a V-bottom plate and washed and stained for FACs analysis. Briefly, cells were stained in 1× Live/Dead BV510 for 15 minutes, washed, and then surface stained with the following antibodies: CD8-BV785 (or CD3-BV605) and CD137-PECy5 (diluted 1:100). Cells were then washed and fixed in 2% paraformaldehyde (PFA). Cells were run on the BD FACSCelesta and FCS files were analyzed in FlowJo. Cells were gated on Single/Live/Lymphocytes/CD8+(or CD3+)/CD137+ or CD137−. The FITC geometric mean of the CD137+ population is shown.

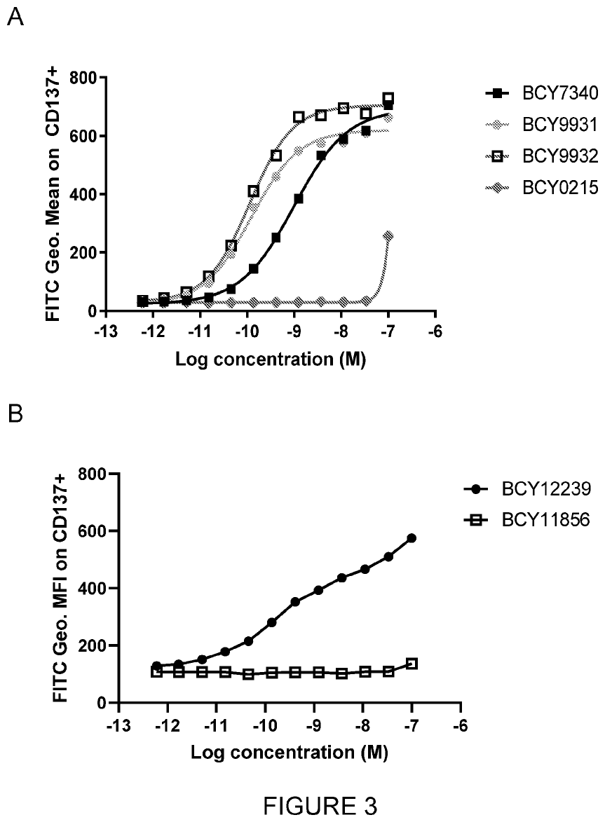

The data shown in FIG. 3A shows that the fluorescent CD137 multimers (BCY7340, BCY9931, and BCY9932) bind to human CD137+ T cells in a dose responsive manner, while the fluorescent EphA2 monomer (BCY0215) does not bind. The fluorescent CD137 dimer (BCY7340) shows slightly less potent binding than the fluorescent trimer (BCY9932) and tetramer (BCY9931 The data in FIG. 3B demonstrates dose responsive binding of the fluorescent CD137 multimer (BCY12239) to human CD137+ T cells. The non-binding version of the fluorescent CD137 multimer (BCY11856) does not bind.

### 3. Fluorescent Multimer Cyno Immune Cell Direct Binding Assay

Cryopreserved PBMCs were purchased from Worldwide Primates and thawed according to standard protocol. CD8+ T cells were then isolated from the PBMCs with a MACS CD8+ T cell isolation kit (negative bead selection) according to the manufacturer's protocol (Miltenyi Biotec). Purified CD8+ T cells were then activated for 48 hours 96 well plates coated with CD3 antibody (1 μg/ml) in R10 media (RPMI1640+10% FBS+1× Pen-Strep). Cells were incubated in R10 media in the presence of fluorescent CD137 multimers or a fluorescent EphA2 bicyclic peptide monomer (BCY0215), which was used as a negative control, at different concentrations for 1 hour at 37° C. After 1 hour, cells were transferred to a V-bottom plate and washed and stained for FACs analysis. Briefly, cells were stained in 1× Live/Dead BV510 for 15 minutes, washed, and then surface stained with the following antibodies: CD8-BV785 and CD137-PECy5 (diluted 1:100 in 2% FBS/PBS). Cells were then washed and fixed in 2% paraformaldehyde (PFA). Cells were run on the BD FACSCelesta and FCS files were analyzed in FlowJo. Cells were gated on Single/Live/Lymphocytes/CD8+/CD137+ or CD137−. The FITC geometric mean of the CD137+ population is shown.

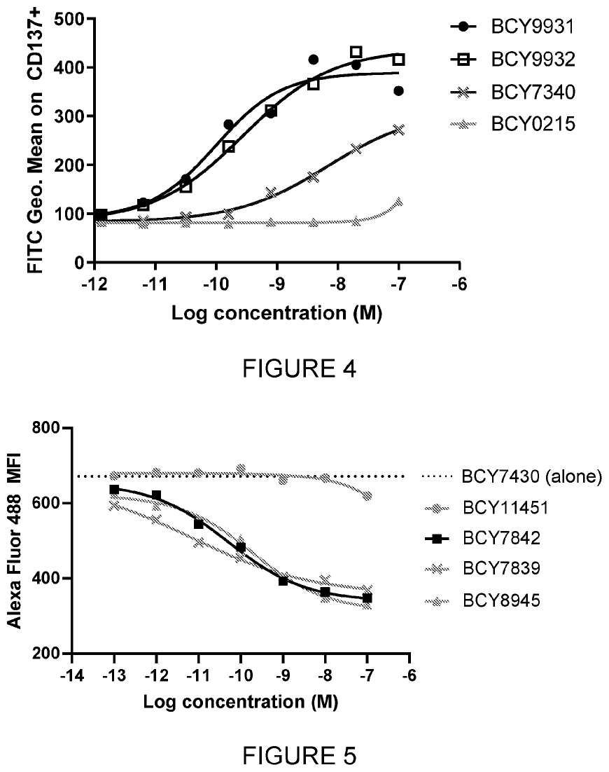

The data shown in FIG. 4 shows that the fluorescent CD137 multimers (BCY7340, BCY9931, and BCY9932) bind to cyno CD137+ T cells in a dose responsive manner, while the fluorescent EphA2 monomer (BCY0215) does not bind. The fluorescent CD137 dimer (BCY7340) shows less potent binding than the fluorescent trimer (BCY9932) and tetramer (BCY9931).

### 4. Fluorescent Multimers Human T-Cell Competition Assay Using Non-Modified Multimeric Bicyclic Peptides BCY8945 and BCY7842

Human PBMCs were isolated from fresh buffy coats from two donors according to standard protocols. CD8+ T cells were then isolated from the PBMCs by negative magnetic bead selection with a MACS CD8+ T cell isolation kit (negative bead selection) according to the manufacturer's protocol (Miltenyi Biotec). Purified CD8+ T cells were then stimulated for 72 hours in plates coated with CD3 antibody (1 μg/ml) in R10 media (RPMI1640+10% FBS+1× Pen-Strep). Cells were then incubated with a range of concentrations of unlabelled CD137 multimer for 30 minutes at room temperature in 2% FBS. Cells were then washed and stained for FACS analysis. Briefly, cells were stained in 1× Live/Dead BV510 for 15 minutes, washed, and then surface stained with a primary antibody cocktail (CD8-BV785, CD3-BV605, and CD137-PECy5) that included a saturating concentration (10 nM) of BCY7340. Cells were incubated for 30 minutes at room temperature, washed, and fixed in 2% PFA. Cells were run on the BD FACSCelesta and FCS files were analyzed in FlowJo. Cells were gated on Single/Live/Lymphocytes/CD8+(or CD3+)/CD137+ or CD137−. The Alexa Fluor 488 geometric mean of the CD137+ population is shown.

In FIG. 5, the fluorescent CD137 multimer (BCY7340) is used as a labelling reagent to measure the level of free/unbound CD137 receptor on human T cells. As unlabelled CD137 multimers (BCY7839, BCY7842, and BCY8945) are titrated, there is a dose responsive decrease in unbound CD137 receptor (and BCY7340 binding). BCY11451 is a non-binding CD137 multimer, therefore the CD137 receptor remains unbound and the fluorescent CD137 multimer binding remains high.

### 5. BCY15416 Direct Binding to CD4 and CD8 Positive T-Cells

To evaluate the affinity of a CD137 dimeric Bicycle® peptide conjugated to Alexa Fluor® 488, herein referred to as BCY15416, a binding assay utilizing CD3 stimulated primary peripheral blood mononuclear cells (PBMCs) was implemented.

On the day of the experiment, medium was prepared by supplementing RPMI-1640 (Gibco™ 11875-093; with L-glutamine) with 10% heat-inactivated fetal bovine serum (FBS; Corning® 35-011-CV), 10 mM HEPES (Gibco™ 15-630-080), and 1% Penicillin Streptomycin (Corning™ 30-002-CI), herein referred to as working medium. Previously isolated human peripheral blood mononuclear cells (PBMCs), from whole blood, were quick thawed in a water bath and washed once at 500 rpm for 5 minutes in 10 mL of prewarmed working medium. PBMC pellet was then resuspended in working medium at a concentration of 3×10⁶ cells/mL. Subsequently, 100 μL of cell suspension was plated in a flat-bottom tissue-culture coated 96-well plate (Greiner CellStar® 655180). Anti-human CD3 (200 ng/mL; BioLegend® 317347; clone OKT3) was added to the cell plate (100 μL/well) at a final concentration of 100 ng/mL Whereas, for unstimulated controls 100 μL of working medium was added. Cells were incubated overnight (12-24 hours) at 37° C., 5% CO₂.

Post-overnight incubation, BCY15416 diluted in working medium was added to the PBMC cell plate at a suggested starting concentration of 300 nM titrated in a % dilution series to perform a 12-point serial dilution. Plates were then incubated for 1 hour at 37° C., 5% CO₂. Post-incubation, plate was centrifuged at 500 rpm for 5 minutes and supernatant discarded. Samples were then washed once in 200 μL of 1× phosphate buffer saline (PBS; Gibco™ 10-010-023) at 500 rpm for 5 minutes. Cells were resuspended in 200 μL of PBS and transferred to a 96-well V-bottom polypropylene plate (Greiner Bio-One 651201). Samples were then centrifuged at 500 rpm for 5 minutes and supernatant was discarded.

Preparation of samples for flow cytometry: Zombie Violet™ Fixable Viability Dye (BioLegend® 423113) was prepared as a 1:1000 dilution in PBS and 100 μL of viability dye was added to each well and incubated in the dark at 4° C. for 30 minutes. Subsequently, wells were washed with 100 μL of PBS for 5 minutes at 500 rpm and supernatant was discarded. Next, human TruStain FcX™ block (BioLegend® 422302) was prepared by diluting 1.5 μL of FcX in 25 μL of stain buffer (1×PBS supplemented with 2% FBS). Fc block solution (25 μL/well) was incubated at room temperature (RT) for 10 minutes in the dark. Antibody master mix cocktail was prepared by diluting 1.5 μL the following antibodies per 100 μL of stain buffer: Brilliant Violet 605™ anti-human CD4 (BD Horizon™ 563875; clone SK3), Brilliant Violet 785™ anti-human CD8a (BioLegend® 301046; clone RPA-T8), and PE/Cyanine5 anti-human CD137 (BioLegend® 309808; clone 4B4-1). Cells were resuspended in master mix cocktail (100 μL) and incubated at 4° C. for 30 minutes in the dark. Subsequently, cells were washed 3 times in 100 μL of stain buffer for 5 minutes at 500 rpm and supernatant was discarded. Cells resuspended in 200 μL of stain buffer were kept at 4° C. and in the dark until read by BD FACSCelesta™ flow cytometer and FCS files were analyzed in FlowJo™.

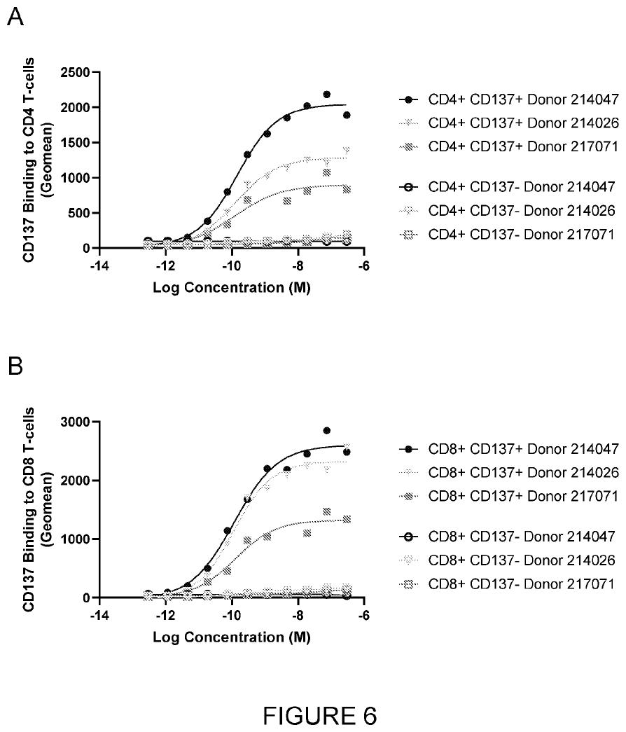

Cells were gated on lymphocytes→singlets→live→CD4+ or CD8+→CD137+/− and the geometric mean of Alexa Fluor® 488 (conjugated to BCY15416) was calculated from the CD137+ and CD137− populations shown below. FIG. 6 illustrates BCY15416 binding exclusively to the CD137+CD4+ T-cells and CD137+CD8+ T-cells, in a dose-dependent manner across three human PBMC donors. Calculated binding affinity (kd,app) for each donor appear in Table 5. The binding affinity of BCY15416 was elucidated to be in the sub-nanomolar range for both CD137+CD4+ and CD137+CD8+ populations. However, in the CD137− populations for both CD4+ and CD8+a binding affinity was unable to be calculated.

TABLE 5
kd, app values of CD137 dimer (BCY15416) were calculated using a
log(agonist) vs response four-parameter variable slope in the CD4+
CD137+ and CD8+ CD137+ populations for all donors

| | Binding affinity | | | |
|---|---|---|---|---|
| | k_{d,app} (nM) | | | |
| | CD4+ | | CD8+ | |
| Donor | CD137+ | CD4+ CD137− | CD137+ | CD8+ CD137− |
| 214047 | 0.1445 | n/a | 0.1183 | n/a |
| 214026 | 0.8754 | n/a | 0.1143 | n/a |
| 217071 | 0.1303 | n/a | 0.1297 | n/a |

FIGS. 6A and 6B demonstrate that BCY15416 is able to bind specifically to CD137+CD4 and CD8 T cells while it does not bind to CD137− CD4 and CD8 T cells. Hence, it can be used as a tool to measure receptor occupancy of other CD137 binders that bind to the same site as BCY15416.

### 6. Receptor Occupancy of BCY12491 Measured by Using BCY15416 as a Probe

A receptor occupancy assay was developed to evaluate binding of CD137 binding Bicycles® to receptors present on immune cell populations by utilizing a competing CD137 dimeric Bicycle® peptide conjugated to Alexa Fluor® 488, herein referred to as BCY15416 or CD137 dimer.

On the day of the experiment, medium was prepared by supplementing RPMI-1640 (Gibco™ 11875-093; with L-glutamine) with 10% heat-inactivated fetal bovine serum (FBS; Corning® 35-011-CV), 10 mM HEPES (Gibco™ 15-630-080), and 1% Penicillin Streptomycin (Corning™ 30-002-CI), herein referred to as working medium. Previously isolated human peripheral blood mononuclear cells (PBMCs), from whole blood, were quick thawed in a water bath and washed once at 500 rpm for 5 minutes in 10 mL of prewarmed working medium. PBMC pellet was then resuspended in working medium at a concentration of 3×10⁶ cells/mL. Subsequently, 100 μL of cell suspension was plated in a flat-bottom tissue-culture coated 96-well plate (Greiner CellStar® 655180). Anti-human CD3 (200 ng/mL; BioLegend® 317347; clone OKT3) was added to the cell plate (100 μL/well) at a final concentration of 100 ng/mL Whereas, for unstimulated controls 100 μL of working medium was added. Cells were incubated overnight (12-24 hours) at 37° C., 5% CO₂.

Post-overnight incubation, test articles were diluted in working medium was added to the PBMC cell plate at a suggested starting concentration of 300 nM titrated in a ¼ dilution series to perform a 12-point serial dilution. Plates were then incubated 1 hour at 37° C., 5% CO₂. Post-incubation, plate was centrifuged at 500 rpm for 5 minutes and supernatant discarded. Samples were then washed once in 200 μL of 1× phosphate buffer saline (PBS; Gibco™ 10-010-023) at 500 rpm for 5 minutes. Cells were resuspended in 200 μL of PBS and transferred to a 96-well V-bottom polypropylene plate (Greiner Bio-One 651201).

Samples were then centrifuged at 500 rpm for 5 minutes and supernatant was discarded. Preparation of samples for flow cytometry: Zombie Violet™ Fixable Viability Dye (BioLegend® 423113) was prepared as a 1:1000 dilution in PBS and 100 μL of viability dye was added to each well and incubated in the dark at 4° C. for 30 minutes. Subsequently, wells were washed with 100 μL of PBS for 5 minutes at 500 rpm and supernatant was discarded. Next, human TruStain FcX™ block (BioLegend® 422302) was prepared by diluting 1.5 μL of FcX in 25 μL of stain buffer (1×PBS supplemented with 2% FBS). Fc block solution (25 μL/well) was incubated at room temperature (RT) for 10 minutes in the dark. Antibody master mix cocktail was prepared by diluting 1.5 μL the following antibodies per 100 μL of stain buffer: Brilliant Violet 605™ anti-human CD4 (BD Horizon™ 563875; clone SK3), Brilliant Violet 785™ anti-human CD8a (BioLegend® 301046; clone RPA-T8), and PE/Cyanine5 anti-human CD137 (BioLegend® 309808; clone 4B4-1). Additionally, 1 nM final concentration of BCY15416 or CD137 dimer was added to the master mix cocktail. Cells were resuspended in master mix cocktail (100 μL) and incubated at 4° C. for 30 minutes in the dark. Subsequently, cells were washed 3 times in 100 μL of stain buffer for 5 minutes at 500 rpm and supernatant was discarded. Cells resuspended in 200 μL of stain buffer were kept at 4° C. and in the dark until read by BD FACSCelesta™ flow cytometer and FCS files were analyzed in FlowJo™.

Flow data is acquired in .fcs format file. Each .fcs file represents one unique sample or well on a 96-well plate. Software FlowJo™ was used to analyze the flow cytometry data. Flow analysis shown below is a representation of flow data analysis for the panel used in this assay:

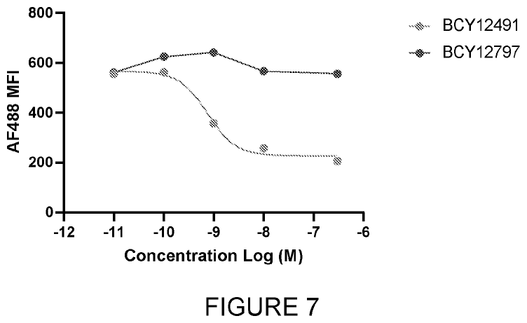

Mean Fluorescence Intensity (MFI) of CD137 Dimer-AF488 positive (high) cells from a subset of total CD137 (PE CY5)+ and total CD137 (PE CY5)−cells is taken was plotted vs concentration of BCY12491. FIG. 7 demonstrates ability of BCY15416 as a probe to determine receptor occupancy of CD137 binding heterotandems such as BCY12491.

1. A multimeric binding complex which comprises at least two bicyclic peptide ligands, wherein said peptide ligands may be the same or different, each of which comprises a polypeptide comprising at least three reactive groups, separated by at least two loop sequences, and a molecular scaffold which forms covalent bonds with the reactive groups of the polypeptide such that at least two polypeptide loops are formed on the molecular scaffold, characterised in that said multimeric binding complex additionally comprises a modifier group conjugated thereto.

2. The multimeric binding complex according to claim 1, wherein the modifier group comprises a tracer molecule, a detectable moiety or a lipid.

3. The multimeric binding complex according to claim 2, wherein the tracer molecule is a fluorophore selected from fluorescein, Alexa Fluor™ 488, cyanine-5 and BODIPY™ FL.

4. The multimeric binding complex according to claim 2, wherein the detectable moiety is a binding detectable moiety, such as a biotin containing moiety, in particular a biotin containing and pegylated moiety, e.g. Biotin-Peg4 and Biotin-Peg12.

5. The multimeric binding complex according to claim 2, wherein the lipid is a palmitoyl containing moiety.

6. The multimeric binding complex according to any one of claims 1 to 5, which comprises a compound of formula (I): 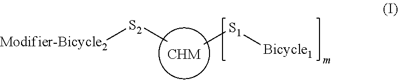
wherein CHM represents a central hinge moiety;
S₁ and S₂ represent spacer groups;
Bicycle₁ and Bicycle₂ represent bicyclic peptide ligands as defined in claim 1;
m represents an integer selected from 1 to 9; and
Modifier represents the modifier group as defined in any one of claims 1 to 5.

7. The multimeric binding complex according to any one of claims 1 to 5, which comprises a compound of formula (II): 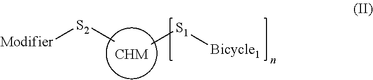
wherein CHM represents a central hinge moiety;
S₁ and S₂ represent spacer groups;
Bicycle₁ represents a bicyclic peptide ligand as defined in claim 1;
n represents an integer selected from 2 to 10; and
Modifier represents the modifier group as defined in any one of claims 1 to 5.

8. The multimeric binding complex according to claim 6 or claim 7, wherein, m and n represent an integer selected from 2 to 9, such as 2 or 3.

9. The multimeric binding complex according to claim 8, wherein m and n represent 3 and CHM is a motif of formula (A): 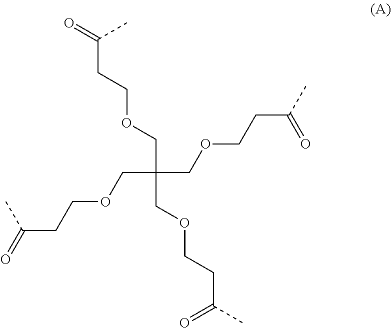
wherein “-----” represents the point of attachment to each spacer group (S₁ or S₂).

10. The multimeric binding complex according to claim 8, wherein n represents 3 and CHM is a motif of formula (B): 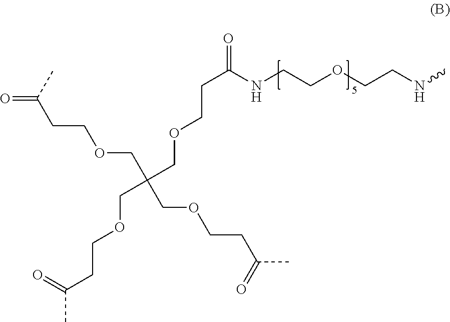
wherein “-----” represents the point of attachment to the spacer group; and
“ [symbol] ” represents the point of attachment to the modifier group.

11. The multimeric binding complex according to claim 8, wherein n represents 2 and CHM is a motif of formula (C): 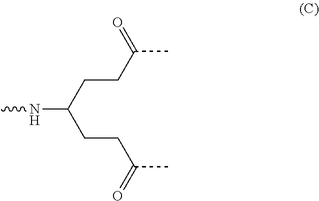
wherein “-----” represents the point of attachment to the spacer group; and
“ [symbol] ” represents the point of attachment to the modifier group.

12. The multimeric binding complex according to claim 8, wherein n represents 2 and CHM is a motif of formula (D): 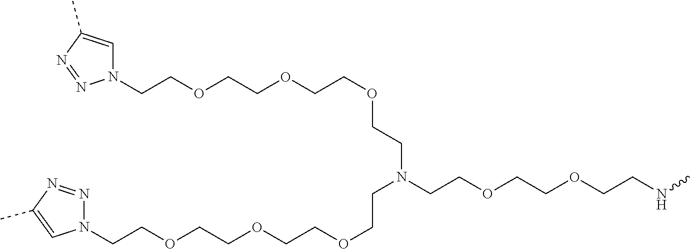
wherein “-----” represents the point of attachment to the spacer group; and
“ [symbol] ” represents the point of attachment to the modifier group.

13. The multimeric binding complex according to any one of claims 6 to 12, wherein the spacers (S₁ and S₂) are selected from any one of spacers S_{A}, S_{B}, S_{C}, S_{D}, S_{E}, S_{F}, S_{G} and S_{H}: 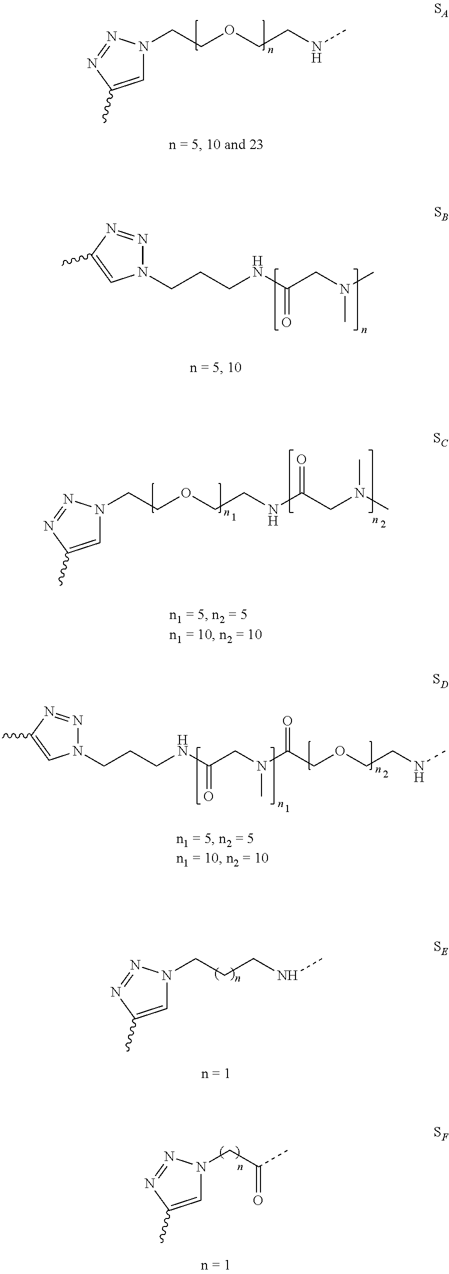 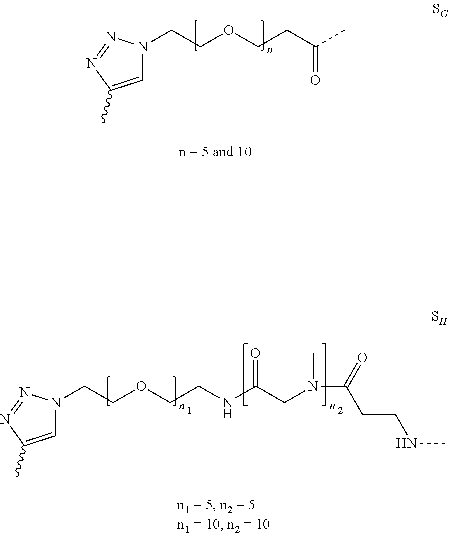
wherein “-----” represents the point of attachment to the CHM group; and
“ [symbol] ” represents the point of attachment to the Bicycle or modifier group, such as S_{A}, wherein n is 5, 10 or 23, such as 10 or 23.

14. The multimeric binding complex according to any one of claims 6 to 12, wherein the spacers (S₁ and S₂) are absent.

15. The multimeric binding complex as defined in any one of claims 1 to 14, wherein at least one of said peptide ligands are specific for CD137, such as each of said peptide ligands are specific for CD137.

16. The multimeric binding complex as defined in any one of claims 1 to 15, wherein said loop sequences comprise 6 amino acid acids.

17. The multimeric binding complex as defined in any one of claims 1 to 16, wherein said peptide ligand comprises a core amino acid sequence selected from:
(SEQ ID NO: 1)
CᵢIEEGQYCᵢᵢFADPY[Nle]Cᵢᵢᵢ;
(SEQ ID NO: 2)
CᵢIEEGQYCᵢᵢFADPYMCᵢᵢᵢ;
(SEQ ID NO: 3)
CᵢIEE[dK(PYA)]QYCᵢᵢFADPY[Nle]Cᵢᵢᵢ;
(SEQ ID NO: 4)
[dCᵢ][dI][dE][dE]K[dQ][dY][dCᵢᵢ][dF][dA][dD][dP]
[dY][dNle][dCᵢᵢᵢ];
(SEQ ID NO: 5)
[dCᵢ][dI][dE][dE]K(PYA)[dQ][dY][dCᵢᵢ][dF][dA][dD]
[dP][dY][dNle][dCᵢᵢᵢ];
(SEQ ID NO: 6)
Cᵢ[tBuAla]PK(PYA)[dA]PYCᵢᵢFADPY[Nle]Cᵢᵢᵢ;
and
(SEQ ID NO: 7)
Cᵢ[tBuAla]PE[D-Lys(PYA)]PYCᵢᵢFADPY[Nle]Cᵢᵢᵢ;
wherein Cᵢ, Cᵢᵢ and Cᵢᵢᵢ represent first, second and third cysteine residues, respectively, Nle represents norleucine, PYA represents propargyl-acid and tBuAla represents t-butyl-alanine, or a pharmaceutically acceptable salt thereof.

18. The multimeric binding complex as defined in claim 17, wherein said peptide ligand comprises N and C terminal additions and comprises an amino acid sequence selected from:
Ac-A-(SEQ ID NO: 1)-[Dap(PYA)]-CONH₂ (hereinafter referred to as BCY7741);
Ac-A-(SEQ ID NO: 1)-[Dap(Lys(PYA))]-CONH₂ (hereinafter referred to as BCY12799);
Ac-(SEQ ID NO: 2)-A-Pra-CONH₂ (hereinafter referred to as BCY7077);
Ac-A-(SEQ ID NO: 3)-A-CONH₂ (hereinafter referred to as BCY7744);
Ac-A-(SEQ ID NO: 3)-K—CONH₂ (hereinafter referred to as BCY11613);
Ac-[dA]-(SEQ ID NO: 4)-[dA]-CONH₂ (hereinafter referred to as BCY11506);
Ac-[dA]-(SEQ ID NO: 5)-[dK]-CONH₂ (hereinafter referred to as BCY12144);
Ac-(SEQ ID NO: 6)-A-CONH₂ (hereinafter referred to as BCY8927);
Ac-(SEQ ID NO: 6)-K—CONH₂ (hereinafter referred to as BCY12357);
Ac-(SEQ ID NO: 7)-A (herein referred to as BCY8928); and
Ac-(SEQ ID NO: 7)-K (herein referred to as BCY13389);
wherein Dap represents diaminopropionic acid, PYA represents propargyl-acid and Pra represents propargylglycine, or a pharmaceutically acceptable salt thereof.

19. The multimeric binding complex as defined in any one of claims 1 to 18, wherein said reactive groups comprise cysteine.

20. The multimeric binding complex as defined in any one of claims 1 to 19, wherein said molecular scaffold is 1,1′,1″-(1,3,5-triazinane-1,3,5-triyl)triprop-2-en-1-one (TATA).

21. The multimeric binding complex as defined in any one of claims 1 to 20 which is listed in Table 1, with the exception of non-binding control BCY12374.

22. The multimeric binding complex as defined in any one of claims 1 to 14, wherein at least one of said peptide ligands (such as two) are specific for CD137 and at least one of said peptide ligands (such as one) is specific for Nectin-4, such as those listed in Table 2.

23. The multimeric binding complex as defined in any one of claims 1 to 14, wherein at least one of said peptide ligands (such as two) are specific for CD137 and at least one of said peptide ligands (such as one) is specific for EphA2, such as those listed in Table 3.

24. The multimeric binding complex as defined in any one of claims 1 to 23, wherein the pharmaceutically acceptable salt is selected from the free acid or the sodium, potassium, calcium, ammonium salt.

25. The multimeric binding complex as defined in any one of claims 13 to 24, wherein the CD137 is human CD137.

26. A drug conjugate comprising the multimeric binding complex as defined in any one of claims 1 to 25, conjugated to one or more effector and/or functional groups.

27. A pharmaceutical composition which comprises the multimeric binding complex of any one of claims 1 to 25 or the drug conjugate of claim 26, in combination with one or more pharmaceutically acceptable excipients.

28. The multimeric binding complex as defined in any one of claims 1 to 25 or the drug conjugate as defined in claim 26, for use in preventing, suppressing or treating a disease or disorder mediated by CD137.

29. Use of a multimeric binding complex as defined in any one of claims 1 to 25, in an analytical method (i.e. as a tracer or a tag).


SEQUENCE LISTING (10 sequences)

SEQ ID NO: 1 - protein, 15 aa, Artificial
  Synthetic Peptide
  Xaa (14)..(14): Xaa is Nle
CIEEGQYCFADPYXC

SEQ ID NO: 2 - protein, 15 aa, Artificial
  Synthetic Peptide
CIEEGQYCFADPYMC

SEQ ID NO: 3 - protein, 15 aa, Artificial
  Synthetic Peptide
  Xaa (5)..(5): Xaa is dK(PYA)
  Xaa (14)..(14): Xaa is Nle
CIEEXQYCFADPYXC

SEQ ID NO: 4 - protein, 15 aa, Artificial
  Synthetic Peptide
  Xaa (14)..(14): Xaa is Nle
CIEEKQYCFADPYXC

SEQ ID NO: 5 - protein, 15 aa, Artificial
  Synthetic Peptide
  Xaa (5)..(5): Xaa is K(PYA)
  Xaa (14)..(14): Xaa is Nle
CIEEXQYCFADPYXC

SEQ ID NO: 6 - protein, 15 aa, Artificial
  Synthetic Peptide
  Xaa (2)..(2): Xaa is tBuAla
  Xaa (4)..(4): Xaa is K(PYA)
  Xaa (14)..(14): Xaa is Nle
CXPXAPYCFADPYXC

SEQ ID NO: 7 - protein, 15 aa, Artificial
  Synthetic Peptide
  Xaa (2)..(2): Xaa is tBuAla
  Xaa (5)..(5): Xaa is D-Lys(PYA)
  Xaa (14)..(14): Xaa is Nle
CXPEXPYCFADPYXC

SEQ ID NO: 8 - protein, 15 aa, Artificial
  Synthetic Peptide
  Xaa (3)..(3): Xaa is 1Nal
  Xaa (7)..(7): Xaa is HArg
  Xaa (13)..(13): Xaa is HyP
CPXDCMXDWSTPXWC

SEQ ID NO: 9 - protein, 15 aa, Artificial
  Synthetic Peptide
  Xaa (2)..(2): Xaa is HyP
  Xaa (14)..(14): Xaa is HArg
CXLVNPLCLHPDWXC

SEQ ID NO: 10 - protein, 15 aa, Artificial
  Synthetic Peptide
  Xaa (2)..(2): Xaa is HyP
  Xaa (12)..(12): Xaa is d1Nal
CXLVNPLCLEPXWTC